(19) United States
(12) Patent Application Publication
(10) Pub. No.: US 2012/0059345 A1
(43) Pub. Date: Mar. 8, 2012

(54) SYSTEMS AND METHODS FOR WASTE DISPOSAL USING A WEARABLE DISPOSAL BAG

(76) Inventors: Douglas C. Dayton, Harvard, MA (US); Sung Park, Chestnut Hill, MA (US)

(21) Appl. No.: 13/291,454

(22) Filed: Nov. 8, 2011

Related U.S. Application Data
(63) Continuation of application No. 12/042,941, filed on Mar. 5, 2008, now abandoned.
Application No. 12/042,941 is a continuation-in-part of application No. 11/738,363, filed on Apr. 20, 2007.
(60) Provisional application No. 60/905,259, filed on Mar. 5, 2007.
(60) Provisional application No. 60/905,151, filed on Mar. 5, 2007.
(60) Provisional application No. 60/905,503, filed on Mar. 6, 2007.
(60) Provisional application No. 61/016,741, filed on Dec. 26, 2007.
(60) Provisional application No. 60/796,839, filed on May 1, 2006.
(60) Provisional application No. 60/797,530, filed on May 3, 2006.
(60) Provisional application No. 60/832,739, filed on Jul. 20, 2006.
(60) Provisional application No. 60/846,449, filed on Sep. 22, 2006.
(60) Provisional application No. 60/846,450, filed on Sep. 22, 2006.
(60) Provisional application No. 60/846,566, filed on Sep. 22, 2006.
(60) Provisional application No. 60/846,565, filed on Sep. 22, 2006.
(60) Provisional application No. 60/847,562, filed on Sep. 26, 2006.
(60) Provisional application No. 60/850,951, filed on Oct. 10, 2006.
(60) Provisional application No. 60/851,372, filed on Oct. 13, 2006.
(60) Provisional application No. 60/852,784, filed on Oct. 18, 2006.
(60) Provisional application No. 60/852,831, filed on Oct. 18, 2006.
(60) Provisional application No. 60/854,222, filed on Oct. 24, 2006.
(60) Provisional application No. 60/854,319, filed on Oct. 24, 2006.
(60) Provisional application No. 60/854,408, filed on Oct. 24, 2006.
(60) Provisional application No. 60/872,269, filed on Dec. 1, 2006.
(60) Provisional application No. 60/854,970, filed on Oct. 26, 2006.
(60) Provisional application No. 60/857,926, filed on Nov. 10, 2006.
(60) Provisional application No. 60/861,338, filed on Nov. 27, 2006.
(60) Provisional application No. 60/872,193, filed on Dec. 1, 2006.
(60) Provisional application No. 60/872,235, filed on Dec. 1, 2006.
(60) Provisional application No. 60/872,192, filed on Dec. 1, 2006.
(60) Provisional application No. 60/872,268, filed on Dec. 1, 2006.
(60) Provisional application No. 60/872,233, filed on Dec. 1, 2006.
(60) Provisional application No. 60/902,921, filed on Feb. 21, 2007.
(60) Provisional application No. 60/902,773, filed on Feb. 21, 2007.

(51) Int. Cl. A61F 13/551

(52) U.S. Cl. 604/385.13

(57) ABSTRACT

In embodiments of the present invention improved capabilities are described for waste disposal. A disposal bag may be configured to receive waste, wherein the bag is wearable. The disposal bag may include a frame associated with the bag at an opening, the frame being configured to enable a user to close the bag in a single-handed operation when opposing sides of the frame are brought into contact with one another and a sealing facility disposed on the frame for providing a resealable closure of the bag when opposing sides of the frame are brought into contact with one another.

### CROSS-REFERENCE TO RELATED APPLICATIONS

This application is a continuation of U.S. patent application Ser. No. 12/042,941 filed Mar. 5, 2008 which is incorporated by reference in its entirety and which claims the benefit of the following provisional applications, each of which is hereby incorporated by reference in its entirety: U.S. provisional patent application Ser. No. 60/905,259, filed Mar. 5, 2007, “FLEXIBLE SNAP TAB REUSABLE CLOSURE WITH PERMANENT ADHESIVE CLOSURE FOR POLYBAG ODOR CONTAINMENT DISPOSAL SYSTEM”; U.S. provisional patent application Ser. No. 60/905,151, filed Mar. 5, 2007, “SAFETY LID FOR THE NESTED POLYBAG DISPENSING CONTAINER”; U.S. provisional patent application Ser. No. 60/905,503, filed Mar. 6, 2007; and U.S. provisional patent application Ser. No. 61/016,741,filed Dec. 26, 2007.

Application Ser. No. 12/042,941 is a continuation-in-part of the following U.S. patent applications, each of which is incorporated by reference in its entirety:

U.S. patent application Ser. No. 11/738,363, filed Apr. 20, 2007, “Systems and Methods for Waste Disposal” which claims the benefit of the following provisional applications, each of which is hereby incorporated by reference in its entirety: U.S. provisional patent application Ser. No. 60/796,839, filed May 1, 2006, “CONTAINMENT SYSTEM FOR DIAPERS AND OTHER ODIFEROUS, MESSY OR HAZARDOUS MATERIALS”; U.S. provisional patent application Ser. No. 60/797,530, filed May 3, 2006, “CONTAINMENT SYSTEM FOR DIAPERS AND OTHER ODIFEROUS, MESSY OR HAZARDOUS MATERIALS”; U.S. provisional patent application Ser. No. 60/832,739, filed Jul. 20, 2006, “CONTAINMENT SYSTEM FOR DIAPERS AND OTHER ODIFEROUS, MESSY OR HAZARDOUS OUT GASSING MATERIALS”; U.S. provisional patent application Ser. No. 60/846,449, filed Sep. 22, 2006, “DISPOSAL SYSTEM FOR DIAPERS AND OTHER ODIFEROUS MATERIALS WITH DUAL ADHESIVE SEALING MECHANISM”; U.S. provisional patent application Ser. No. 60/846,450, filed Sep. 22,2006, “DISPOSAL SYSTEM WITH SCENTED ELEMENT FOR DISPOSING OF DIAPERS AND OTHER ODIFEROUS MATERIALS”; U.S. provisional patent application Ser. No. 60/846,566, filed Sep. 22, 2006, “DISPOSAL SYSTEM HAVING NESTED STACK OF SEALABLE ENCLOSURES FOR DISPOSING OF DIAPERS AND OTHER ODIFEROUS MATERIALS”; U.S. provisional patent application Ser. No. 60/846,565, filed Sep. 22, 2006, “FLANGE APPARATUS FOR DISPOSAL SYSTEM FOR DISPOSING OF DIAPERS AND OTHER ODIFEROUS MATERIALS”; U.S. provisional patent application Ser. No. 60/847,562, filed Sep. 26, 2006, “BAG OR CONTAINER FOR DISPOSING OF DIAPERS AND OTHER ODIFEROUS MATERIALS”; U.S. provisional patent application Ser. No. 60/850,951, filed Oct. 10, 2006, “DISPOSAL SYSTEM WITH INTEGRAL CLOSURE ELEMENT”; U.S. provisional patent application Ser. No. 60/851,372, filed Oct. 13, 2006, “DISPOSAL SYSTEM FOR DIAPERS AND OTHER ODIFEROUS MATERIALS WITH BAG EXTENSION”; U.S. provisional patent application Ser. No. 60/852,784, filed Oct. 18, 2006, “DISPOSAL SYSTEM FOR DIAPERS AND OTHER ODIFEROUS MATERIALS WITH ROUNDED SEALABLE FLANGE MEMBER”; U.S. provisional patent application Ser. No. 60/852,831, filed Oct. 18, 2006, “NESTED AND STACKED BAG DISPENSER FOR DISPOSAL OF DIAPERS AND OTHER ODIFEROUS MATERIALS”; U.S. provisional patent application Ser. No. 60/854,222, filed Oct. 24, 2006, “SYSTEM WITH INTEGRATED TISSUE DISPENSER FOR DISPOSAL OF DIAPERS AND OTHER ODIFEROUS MATERIALS”; U.S. provisional patent application Ser. No. 60/854,319, filed Oct. 24, 2006, “DIAPER DISPOSAL SYSTEM WITH ATTACHED BAG”; U.S. provisional patent application Ser. No. 60/854,408, filed Oct. 24, 2006, “DISPOSAL SYSTEM WITH MOLDED CLOSURE FLANGE”; U.S. provisional patent application Ser. No. 60/872,269, filed Dec. 1, 2006; “DISPOSAL SYSTEM WITH MOLDED CLOSURE FLANGE HAVING DISCRETE INTERLOCKS”; U.S. provisional patent application Ser. No. 60/854,970, filed Oct. 26, 2006, “DISPOSAL SYSTEM WITH VENTED BAG”; U.S. provisional patent application Ser. No. 60/857,926, filed Nov. 10, 2006, “DISPOSAL SYSTEM HAVING ENCLOSURES WITH PROTECTED RIMS”; U.S. provisional patent application Ser. No. 60/861,338, filed Nov. 27, 2006, “REFILLABLE BAG DISPOSAL SYSTEM”; provisional patent application Ser. No. 60/872,193, filed Dec. 1, 2006, “DISPOSAL SYSTEM WITH MOLDED CLOSURE FLANGE HAVING DISCRETE INTERLOCKS”; U.S. provisional patent application Ser. No. 60/872,235, filed Dec. 1, 2006, “DISPOSAL SYSTEM WITH SECURING APPARATUS”; U.S. provisional patent application Ser. No. 60/872,192, filed Dec. 1, 2006, “DISPOSAL SYSTEM COMPONENTS INTEGRATED IN FRAME”; U.S. provisional patent application Ser. No. 60/872,268, filed Dec. 1, 2006, “DISPOSAL BAG WITH PLUGGABLE VENT”; U.S. provisional patent application Ser. No. 60/872,233, filed Dec. 1, 2006, “DISPOSAL CONTAINER WITH OPPOSING MECHANICAL CLOSURE MEMBERS”; U.S. provisional patent application Ser. No. 60/902,921, filed Feb. 21, 2007, “DISPOSAL SYSTEM WITH ATTACHED BAG (II)”; and U.S. provisional patent application Ser. No. 60/902,773, filed Feb. 21, 2007, “DISPOSAL SYSTEM WITH SNAP TAB CLOSURE”.

### BACKGROUND

1. Field

This invention generally relates to methods and systems of waste disposal, and specifically relates to a waste disposal system for the air-tight and odor-free disposal of waste.

2. Description of the Related Art

Numerous household and industrial processes may generate odiferous and untidy waste whose disposal poses a problem because of the high odor that emanates from the refuse as it sits in a trash container over time and the untidy conditions of disposal when such disposal is not hands free. Existing disposal systems may be cumbersome to operate and may not contain odors.

A need exists for improved methods and systems for disposal of odiferous or messy waste, including methods and systems for disposal of odiferous waste by a hands-free operation.

### SUMMARY

Provided herein may be methods and systems for waste disposal. In an aspect of the invention, a disposal bag comprises a bag configured to receive waste, a frame attached to the bag at an opening, the frame being configured to enable a user to close the bag in a single-handed operation when opposing sides of the frame are brought into contact with one another, and a sealing facility disposed on the frame for providing a resealable closure of the bag when opposing sides of the frame are brought into contact with one another. In an embodiment, the bag is configured to be detachably attached to a diaper. In an embodiment, the bag is glove-shaped. In an embodiment, the bag is free-standing. In an embodiment, the frame is hinged. In an embodiment, the frame of a first disposal bag provides support on its upper surface for the frame of a second disposal bag when a second bag is positioned on top of the first bag such that the lower surface of the second bag's frame rests on the upper surface of the first bag's frame and the second bag extends into the first bag. In an embodiment, sealing facility is at least one of a mechanical, chemical, magnetic, thermal, adhesive, molecular, electrical, electromagnetic, electrostatic, nanofiber, and nanostructure-based connection. In an example of this embodiment, the adhesive is reusable. In an example of this embodiment, the adhesive is permanent. In an embodiment, the sealing facility is at least one of an interference locking system, a tab, a ball closure, discrete interlocks, a snap fit, a clasp, a clip, a zipper, a snap, a gasket, an O-ring type closure, a hook-and-loop, a hook-and-eye, and a spring-locking hinge. In an embodiment, the disposal bag further comprises a gasket mounted to the frame. In an embodiment, the disposal bag further comprises a release facility disposed on the disposal bag.

In an aspect of the invention, a method of providing a disposal bag comprises configuring a bag to receive waste, attaching a frame to the bag at an opening, the frame being configured to close the bag in a single-handed operation when opposing sides of the frame are brought into contact with one another, and disposing a sealing facility on the frame for providing a resealable closure of the bag when opposing sides of the frame are brought into contact with one another. In an embodiment, the bag is configured to be detachably attached to a diaper. In an embodiment, the bag is glove-shaped. In an embodiment, bag is free-standing. In an embodiment, the frame is hinged. In an embodiment, the frame of a first disposal bag provides support on its upper surface for the frame of a second disposal bag when a second bag is positioned on top of the first bag such that the lower surface of the second bag's frame rests on the upper surface of the first bag's frame and the second bag extends into the first bag. In an embodiment, the sealing facility is at least one of a mechanical, chemical, magnetic, thermal, adhesive, molecular, electrical, electromagnetic, electrostatic, nanofiber, and nanostructure-based connection. In an example of this embodiment, the adhesive is reusable. In an example of this embodiment, adhesive is permanent. In an embodiment, the sealing facility is at least one of an interference locking system, a tab, a ball closure, discrete interlocks, a snap fit, a clasp, a clip, a zipper, a snap, a gasket, an O-ring type closure, a hook-and-loop, a hook-and-eye, and a spring-locking hinge. In an embodiment, the method further comprises mounting a gasket to the frame. In an embodiment, the method further comprises disposing a release facility on the disposal bag.

In an aspect of the invention, a disposal bag comprises a bag configured to receive waste, a frame attached to the bag at an opening, the frame being configured to close the bag when opposing sides of the frame are brought into contact with one another, and a release facility disposed on at least one of the frame and the bag to remove excess volume of material from a sealed bag. In an embodiment, the release facility is a venting facility. In an embodiment, the release facility is a valve. In an embodiment, the excess volume is in vapor form. In an embodiment, the excess volume is in gas form. In an embodiment, the excess volume is in liquid form. In an embodiment, the release facility is at least one of a one-way vent, a two-way vent, a channel formed when opposing sides of the frame are brought into contact with one another, a channel formed on a single side of the frame, a gasket, a valve, and a vent tube removably affixed to the frame. In an embodiment, the release facility is mounted in the bag material. In an embodiment, the release facility is at least one of sealed, self-sealed, and plugged after the disposal bag is vented. In an embodiment, the bag is configured to be detachably attached to a diaper. In an embodiment, the bag is glove-shaped. In an embodiment, the bag is free-standing. In an embodiment, the frame is hinged. In an embodiment, a first disposal bag is configured to be nested in a second disposal bag. In an embodiment, the disposal bag further comprises a sealing facility disposed on the frame. In an example of this embodiment, the sealing facility is at least one of a mechanical, chemical, magnetic, thermal, adhesive, molecular, electrical, electromagnetic, electrostatic, nanofiber, and nanostructure-based connection. In embodiments, the adhesive is reusable. In embodiments, the adhesive is permanent. In an example of this embodiment, the sealing facility is at least one of an interference locking system, a tab, a ball closure, discrete interlocks, a snap fit, a clasp, a clip, a zipper, a snap, a gasket, an O-ring type closure, a hook-and-loop, a hook-and-eye, and a spring-locking hinge. In an embodiment, the disposal bag further comprises a gasket mounted to the frame.

In an aspect of the invention, a method of providing a disposal bag comprises configuring a bag to receive waste, attaching a frame to the bag at an opening, the frame being configured to close the bag when opposing sides of the frame are brought into contact with one another, and disposing a release facility on at least one of the frame and the bag to remove excess volume of material from a sealed bag. In an embodiment, the release facility is a venting facility. In an embodiment, the release facility is a valve. In an embodiment, the excess volume is in vapor form. In an embodiment, the excess volume is in gas form. In an embodiment, the excess volume is in liquid form. In an embodiment, the release facility is at least one of a one-way vent, a two-way vent, a channel formed when opposing sides of the frame are brought into contact with one another, a channel formed on a single side of the frame, a gasket, a valve, and a vent tube removably affixed to the frame. In an embodiment, the release facility is mounted in the bag material. In an embodiment, the release facility is at least one of sealed, self-sealed, and plugged after the disposal bag is vented. In an embodiment, the bag is configured to be detachably attached to a diaper. In an embodiment, the bag is glove-shaped. In an embodiment, the bag is free-standing. In an embodiment, the frame is hinged. In an embodiment, a first disposal bag is configured to be nested in a second disposal bag. In an embodiment, the method further comprises disposing a sealing facility on the frame. In an example of this embodiment, the sealing facility is at least one of a mechanical, chemical, magnetic, thermal, adhesive, molecular, electrical, electromagnetic, electrostatic, nanofiber, and nanostructure-based connection. In embodiments, the adhesive is reusable. In embodiments, the adhesive is permanent. In an example of this embodiment, the sealing facility is at least one of an interference locking system, a tab, a ball closure, discrete interlocks, a snap fit, a clasp, a clip, a zipper, a snap, a gasket, an O-ring type closure, a hook-and-loop, a hook-and-eye, and a spring-locking hinge. In an embodiment, the method further comprises mounting a gasket to the frame.

In an aspect of the invention, a disposal bag comprises a bag configured to receive waste and a frame attached to the bag at an opening, the frame being configured to close the bag when opposing sides of the frame are brought into contact with one another, wherein the frame of a first disposal bag provides support on its upper surface for the frame of a second similar disposal bag when a second bag is positioned on top of the first bag such that the lower surface of the second bag's frame rests on the upper surface of the first bag's frame and the second bag extends into the first bag. In an embodiment, the disposal bag is used in a hands-free operation. In an embodiment, the bag is glove-shaped. In an embodiment, the bag is free-standing. In an embodiment, the frame is hinged. In an embodiment, closure of the frame is configured for single hand operation. In an embodiment, the disposal bag further comprises a sealing facility disposed on the frame. In an example of this embodiment, the sealing facility is at least one of a mechanical, chemical, magnetic, thermal, adhesive, molecular, electrical, electromagnetic, electrostatic, nanofiber, and nanostructure-based connection. In embodiments, the adhesive is reusable. In embodiments, the adhesive is permanent. In an example of this embodiment, the sealing facility is at least one of an interference locking system, a tab, a ball closure, discrete interlocks, a snap fit, a clasp, a clip, a zipper, a snap, a gasket, an O-ring type closure, a hook-and-loop, a hook-and-eye, and a spring-locking hinge. In an embodiment, the disposal bag further comprises a gasket mounted to the frame. In an embodiment, the disposal bag further comprises a release facility disposed on the disposal bag.

In an aspect of the invention, a method of providing a disposal bag comprises configuring a bag to receive waste and attaching a frame to the bag at an opening, the frame being configured to close the bag when opposing sides of the frame are brought into contact with one another, wherein the frame of a first disposal bag provides support on its upper surface for the frame of a second similar disposal bag when a second bag is positioned on top of the first bag such that the lower surface of the second bag's frame rests on the upper surface of the first bag's frame and the second bag extends into the first bag. In an embodiment, the disposal bag is used in a hands-free operation. In an embodiment, the bag is free-standing. In an embodiment, the bag is glove-shaped. In an embodiment, the frame is hinged. In an embodiment, closure of the frame is configured for single hand operation. In an embodiment, the method further comprises disposing a sealing facility on the frame. In an example of this embodiment, the sealing facility is at least one of a mechanical, chemical, magnetic, thermal, adhesive, molecular, electrical, electromagnetic, electrostatic, nanofiber, and nanostructure-based connections. In embodiments, the adhesive is reusable. In embodiments, the adhesive is permanent. In an example of this embodiment, the sealing facility is at least one of an interference locking system, a tab, a ball closure, discrete interlocks, a snap fit, a clasp, a clip, a zipper, a snap, a gasket, an O-ring type closure, a hook-and-loop, a hook-and-eye, and a spring-locking hinge. In an embodiment, the method further comprises mounting a gasket to the frame. In an embodiment, the method further comprises disposing a release facility on the disposal bag.

In an aspect of the invention, a waste disposal system comprises a bag configured to receive waste, a frame attached to the bag at an opening, the frame being configured to close the opening when opposing sides of the frame are brought into contact with one another, a sealing facility disposed on the frame to provide a resealable closure when opposing sides of the frame are brought into contact with one another, an adhesive disposed on at least one side of the frame to provide a permanent seal when opposing sides of the frame are brought into contact with one another, and a container for dispensing a plurality of bags, wherein the bags nest one within the other in the container. In an embodiment, the sealing facility is at least one of a mechanical, chemical, magnetic, thermal, adhesive, molecular, electrical, electromagnetic, electrostatic, nanofiber, and a nanostructure-based connection. In an example of this embodiment, the adhesive is reusable. In an example of this embodiment, the adhesive disposed on the at least one side of the frame is reusable and the adhesive disposed on the opposing side of the frame is permanent. In an embodiment, the sealing facility is at least one of an interference locking system, a tab, a ball closure, discrete interlocks, a snap fit, a clasp, a clip, a zipper, a snap, a gasket, an O-ring type closure, a hook-and-loop, a hook-and-eye, and a spring-locking hinge. In an embodiment, the frame is hinged. In an embodiment, closure of the frame is configured for single hand operation. In an embodiment, waste cleanup articles are positioned in a portion of the container. In an embodiment, the container is refillable. In an embodiment, the container is suspended. In an embodiment, the container is mounted near a location of a waste-making activity. In an embodiment, the waste disposal system further comprises a gasket mounted to the frame. In an embodiment, the waste disposal system further comprises a release facility disposed on the disposal bag.

In an aspect of the invention, a method of providing a waste disposal system comprises configuring a bag to receive waste, attaching a frame to the bag at an opening, the frame being configured to close the opening when opposing sides of the frame are brought into contact with one another, disposing a sealing facility on the frame to provide a resealable closure when opposing sides of the frame are brought into contact with one another, disposing an adhesive on at least one side of the frame to provide a permanent seal when opposing sides of the frame are brought into contact with one another, and providing a container for dispensing a plurality of bags, wherein the bags nest one within the other in the container. In an embodiment, the sealing facility is at least one of a mechanical, chemical, magnetic, thermal, adhesive, molecular, electrical, electromagnetic, electrostatic, nanofiber, and nanostructure-based connection. In an example of this embodiment, the adhesive is reusable. In an example of this embodiment, the adhesive disposed on the at least one side of the frame is reusable and the adhesive disposed on the opposing side of the frame is permanent. In an embodiment, the sealing facility is at least one of an interference locking system, a tab, a ball closure, discrete interlocks, a snap fit, a clasp, a clip, a zipper, a snap, a gasket, an O-ring type closure, a hook-and-loop, a hook-and-eye, and a spring-locking hinge. In an embodiment, the frame is hinged. In an embodiment, closure of the frame is configured for single hand operation. In an embodiment, the method further comprises positioning waste cleanup articles in a portion of the container. In an embodiment, the container is refillable. In an embodiment, the container is suspended. In an embodiment, the container is mounted near a location of a waste-making activity. In an embodiment, the method further comprises mounting a gasket to the frame. In an embodiment, the method further comprises disposing a release facility on the disposal bag.

In an aspect of the invention, a waste container lid comprises an orifice for supporting a disposal bag container, an orifice for supporting waste clean-up articles, and an opening sized to allow articles for disposal to pass through it, including waste-containing disposal bags, wherein the waste container lid is configured to attach to a top portion of a waste container. In an embodiment, the waste container lid further comprises a cover of the waste container lid opening. In an embodiment, the cover is hinged. In an embodiment, the cover is opened by a spring-loaded release mechanism. In an embodiment, the cover is opened by a motion detection system. In an embodiment, the waste container lid further comprises a secondary disposal bag disposed within the waste container sized for receiving sealed disposal bags. In an embodiment, the waste clean-up articles are one or more of wet tissues, dry tissues. hand sanitizer, skin moisturizer, soap, water, cream, lotion, and medication. In an embodiment, the container supports the disposal bag by its frame. In an embodiment, the container dispenses the disposal bag in a pop-up configuration. In an embodiment, the container dispenses the disposal bag in a nesting configuration. In an embodiment, the container dispenses the disposal bag in a stacked configuration.

In an aspect of the invention, a method of providing a waste container lid comprises providing an orifice for supporting a disposal bag container, providing an orifice for supporting waste clean-up articles, and providing an opening sized to allow articles for disposal to pass through it, including waste-containing disposal bags, wherein the waste container lid is configured to attach to a top portion of a waste container. In an embodiment, the method further comprises providing a cover of the waste container lid opening. In an embodiment, the cover is hinged. In an embodiment, the cover is opened by a spring-loaded release mechanism. In an embodiment, the cover is opened by a motion detection system. In an embodiment, the method further comprises disposing a secondary disposal bag within the waste container sized for receiving sealed disposal bags. In an embodiment, the waste clean-up articles are one or more of wet tissues, dry tissues. hand sanitizer, skin moisturizer, soap, water, cream, lotion, and medication. In an embodiment, the container supports the disposal bag by its frame. In an embodiment, the container dispenses the disposal bag in a pop-up configuration. In an embodiment, the container dispenses the disposal bag in a nesting configuration. In an embodiment, the container dispenses the disposal bag in a stacked configuration.

In an aspect of the invention, a disposal bag comprises a bag configured to receive waste and a frame attached to the bag at an opening, the frame being configured to close the bag when opposing sides of the frame are brought into contact with one another. Further, the frame of a first disposal bag provides support on its upper surface for the frame of a second similar disposal bag when a second bag is positioned on top of the first bag such that the lower surface of the second bag's frame fits into the upper surface of the first bag's frame and the second bag extends into the first bag. In an embodiment, the disposal bag is used in a hands-free operation. In an embodiment, the bag is glove-shaped. In an embodiment, the bag is free-standing. In an embodiment, the frame is hinged. In an embodiment, the frame has a conical section. In an embodiment, closure of the frame is configured for single hand operation. In an embodiment, the disposal bag further comprises a sealing facility disposed on the frame. In an example of this embodiment, the sealing facility is at least one of a mechanical, chemical, magnetic, thermal, adhesive, molecular, electrical, electromagnetic, electrostatic, nanofiber, and nanostructure-based connection. In embodiments, the adhesive is reusable. In embodiments, the adhesive is permanent. In an example of this embodiment, the sealing facility is at least one of an interference locking system, a tab, a ball closure, discrete interlocks, a snap fit, a clasp, a clip, a zipper, a snap, a gasket, an O-ring type closure, a hook-and-loop, a hook-and-eye, and a spring-locking hinge. In an embodiment, the disposal bag further comprises a gasket mounted to the frame. In an embodiment, the disposal bag further comprises a release facility disposed on the disposal bag.

In an aspect of the present invention, a disposal bag comprises a bag configured to receive waste and a frame attached to the bag at an opening. The frame includes one or more pair of hinge points configured to open and close the bag when a force is applied on the frame and a pair of tabs associated with one of the one or more pair of hinge points, wherein the pair of tabs enable stacking of the disposal bag. In an embodiment, the disposal bag includes a plurality of vertical legs configured to provide free-standing capability to the disposal bag. In an embodiment, the disposal bag is used in a hands-free operation. In an embodiment, opening and closure of the frame is configured for single hand operation. In an embodiment, the disposal bag further comprises a sealing facility disposed on the frame. In another embodiment, the disposal bag further comprises a sealing facility disposed on the disposal bag. The sealing facility is at least one of a mechanical, chemical, magnetic, thermal, adhesive, molecular, electrical, electromagnetic, electrostatic, nanofiber, and nanostructure-based connection. In embodiments, the adhesive is reusable. In embodiments, the adhesive is permanent. In an example of this embodiment, the sealing facility is at least one of an interference locking system, a tab, a ball closure, discrete interlocks, a snap fit, a clasp, a clip, a zipper, a snap, a gasket, an O-ring type closure, a hook-and-loop, a hook-and-eye, and a spring-locking hinge. In an embodiment, the disposal bag further comprises a gasket mounted to the frame. In an embodiment, the disposal bag further comprises a release facility disposed on the disposal bag.

In embodiments, the disposal bag may be stacked in a dispensing container. The dispensing container may include a plurality of wires configured to enable stacking of the disposal bag in the dispensing container.

These and other systems, methods, objects, features, and advantages of the present invention will be apparent to those skilled in the art from the following detailed description of the preferred embodiment and the drawings. All documents mentioned herein are hereby incorporated in their entirety by reference.

### BRIEF DESCRIPTION OF THE FIGURES

The invention and the following detailed description of certain embodiments thereof may be understood by reference to the following figures:

FIG. 1 depicts a free-standing disposal bag configured as a box.

FIG. 2 depicts the pattern for a free-standing disposal bag configured as a box.

FIG. 3 depicts an unstructured bag with a frame comprising tabs.

FIG. 4A depicts a frame for a disposal bag with an interference locking system; FIG. 4B depicts a frame for a disposal bag with an interference locking system and an alternative gasket solution.

FIG. 5 depicts a disposal bag with a frame formed from the material of the bag.

FIG. 6 depicts a frame for a disposal bag with handles.

FIG. 7 depicts adhesive disposed along the surface of the frame of a disposal bag.

FIG. 8 depicts a ball-type purse closure for a disposal bag frame.

FIG. 9 depicts a disposal bag with extended bag material in the form of a flap.

FIG. 10 depicts a sideways useable system with extended flap allowing for scooping or sweeping waste inside the bag.

FIG. 11A depicts a cross-section of the tissues and their dispenser disposed inside the disposal bag stack when the system is in the closed position; FIG. 11B depicts a cross-section of the system in the open position with both tissues and the disposal bags available for use.

FIG. 12 depicts a peel tab binding for a stack of nested bags.

FIG. 13 depicts a container with a channel.

FIG. 14 depicts a hinged lid with an opening for a disposal bag container.

FIG. 15 depicts a shaped wire for suspending a container of disposal bags.

FIG. 16 depicts a disposal bag system with an orifice for disposal bags, an orifice for a bottle, and an opening for trash.

FIG. 17 depicts a container and lid with mating threads.

FIG. 18 depicts a disposal bag configured to be worn as a glove.

FIG. 19 depicts a disposal bag attached to a diaper.

FIG. 20 depicts a channel formed in a frame with an attached plug.

FIG. 21 depicts a frame with discrete interlocking features.

FIGS. 22-24 depict use of the disposal bag configured as a box.

FIG. 25 depicts a disposal bag with a mechanical fastening at the edge of a frame.

FIG. 26 depicts a disposal bag with adhesive disposed on a frame with an odor reducing agent. The adhesive is not exposed in (A) and exposed in (B), along with the odor reducing agent.

FIGS. 27-32 depicts operation of a disposal bag with a rounded frame.

FIG. 33 depicts a disposal bag with a member containing an odor reducing agent.

FIG. 34 depicts two alternatives of flange design.

FIGS. 35 and 36 depict operation of a disposal bag with adhesive along an integral closure member.

FIG. 37 depicts a sequence of steps for dispensing and using a disposal bag.

FIGS. 38 depicts an exemplary set of steps for using a disposal bag with an extended flap and sealing it.

FIG. 39 depicts operation of a disposal bag with a clasp closure.

FIG. 40 depicts a disposal bag type for a large secondary waste container.

FIGS. 41 and 42 depict a circular frame and closing the frame along a hinge line.

FIG. 43 depicts a series of features of a rounded, resealable frame.

FIGS. 44 and 45 depict a plurality of nested bags within each other and stacked above one another in a dispenser and the container with a cover.

FIG. 46 depicts placing waste into the top-most nested bag with a cover protector around the perimeter of the nested bag frames.

FIG. 47 depicts removing the top-most nested bag from the nested stack using tabs on its closure frame, one-handed closing of the disposal bag, and compressing the excess air from the used bag for compaction prior to storage or disposal.

FIG. 48 depicts a disposal bag system with disposal bags in the portion of the system on the left and tissues in the portion of the system on the right.

FIG. 49 depicts a disposal bag with an exemplary molded frame and closing the open bag with a single hand.

FIG. 50 depicts an exemplary disposal bag with a one-way collapsible valve, a fragrant member near a vent, and a portion of the bag for sealing the valve.

FIG. 51 depicts a disposal system with nested bags and a cross-section of the disposal system.

FIGS. 52-54 depict a refill pack for a disposal bag dispenser.

FIG. 55 depicts a piece of furniture with a container of diposal bags clamped to an edge.

FIG. 56 depicts mounting devices for a container of disposal bags.

FIG. 57 depicts a disposal system.

FIG. 58 depicts opposing ball members that permit shutting and opening a disposal bag and the opposing ball members and an adhesive.

FIG. 59 depicts a flat pack disposal bag with adhesive for attachment to a diaper, and a container for flat pack disposal bags.

FIG. 60 depicts opening the disposal bag attached to the diaper, inserting the soiled diaper into the disposal bag, and the sealed disposal bag with soiled diaper contents.

FIG. 61 depicts opposed snap tabs coming into proximity and finger pressure being applied to deflect the snap tabs.

FIG. 62 depicts opposed snap tabs deflecting past one another and a hinged frame with snap tabs and an adhesive disposed on the frame protected by a peel away layer.

FIG. 63 depicts a sealed disposal bag being vented through a tube in the frame and the tube being removed after venting.

FIG. 64A depicts a sealed, vented disposal bag; FIG. 64B depicts a disposal bag with a hinged frame comprising opposed snap tabs and foam tape.

FIG. 65 depicts sealing a frame by deflecting opposed snap tabs, venting the sealed disposal bag through a tube, and removing the tube from the sealed, vented disposal bag.

FIG. 66 depicts a container with a safety lid.

FIG. 67 depicts a stack of nested bags in a container.

FIG. 68 depicts operation of a disposal bag container lid with spring loaded snap lock engaging features.

FIG. 69A depicts a glove or mitten shaped disposal bag and a frame for attachment to the bag; FIG. 69B depicts grasping waste with a hand placed inside a disposal bag.

FIG. 70A depicts inverting the disposal bag once waste has been placed within the disposal bag; FIG. 70B depicts removing a peel away layer from an adhesive disposed on the frame of the disposal bag.

FIG. 71A depicts shutting a disposal bag; FIG. 71B depicts a sealed disposal bag with a release facility.

FIG. 72A depicts a disposal bag with a conical frame; FIG. 72B depicts the conical frame of the disposal bag; FIG. 72C depicts nesting of the disposal bags with conical frame; FIG. 72D depicts adhesive disposed along the surface of the conical frame of the disposal bag.

FIGS. 73A and 73B depict a disposal bag according to an embodiment of the present invention.

FIGS. 74A and 74B depict a disposal bag according to another embodiment of the present invention.

FIGS. 75A and 75B depict stacking of disposal bags in a dispensing container according to an embodiment of the present invention.

FIGS. 76-78 depict use of a disposal bag according to an embodiment of the present invention.

FIG. 79 depicts use of a disposal bag according to another embodiment of the present invention.

### DETAILED DESCRIPTION

Waste disposal methods and systems involve containment of the materials to be disposed of and may also involve containment of odor, use during waste-making activities, and use in an environmentally sound manner. An improvement that may be provided by the present invention relates to a waste disposal system comprising a disposal bag with a frame and related methods for inexpensive, compact, and convenient disposal of waste in an air-tight, odor-controlled, single-hand operable disposal bag. Waste may be placed in a disposal bag, which may be either free-standing or presented for use in a dispenser, and the frame may be folded at a hinge such that opposing sides of the frame are brought into contact with one another and the waste is sealed within the disposal bag. The frame may provide means for temporary and and/or permanent seal of the disposal bag in a substantially airtight and odor-free manner. The disposal bag may be stored for an extended period of time before final disposal in an odor-free manner.

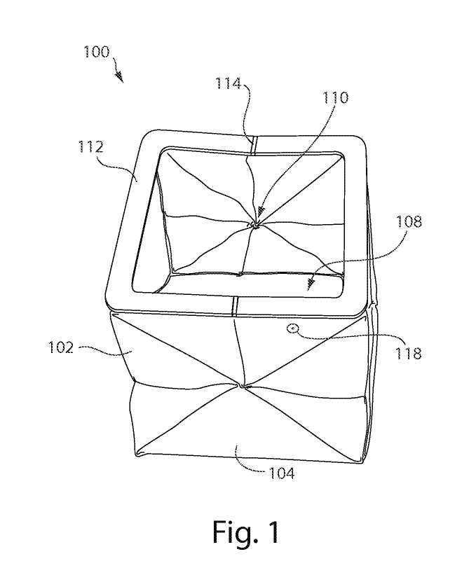

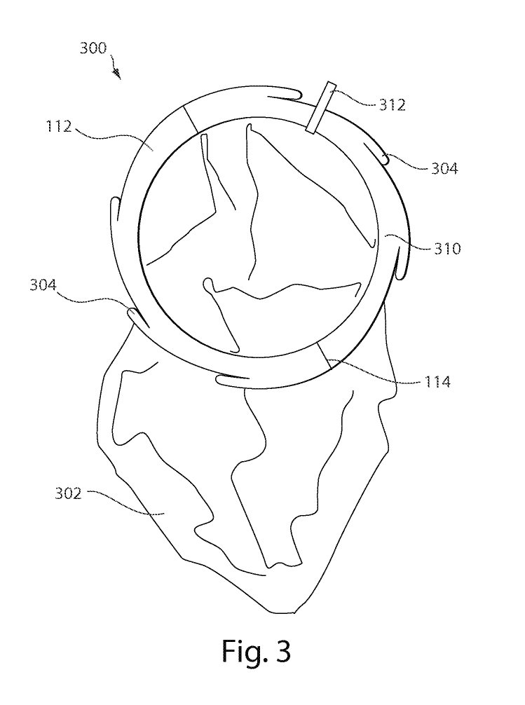

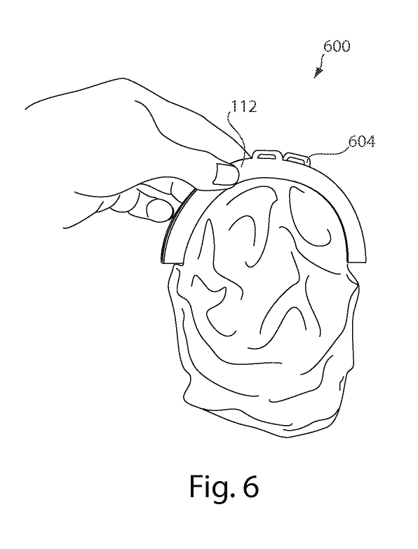

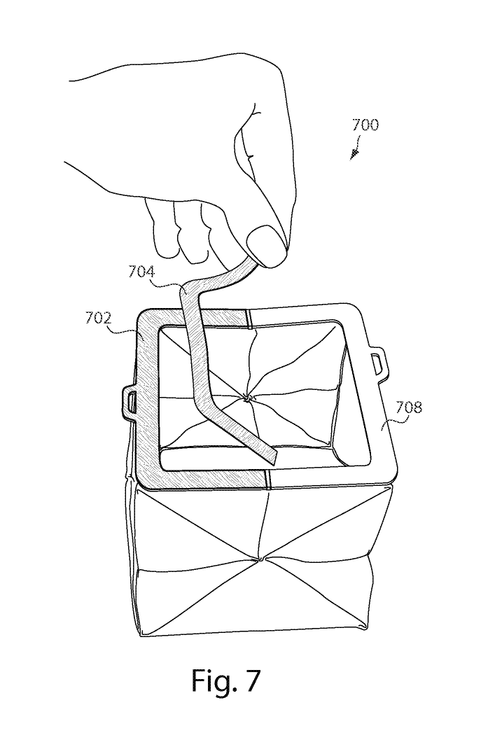

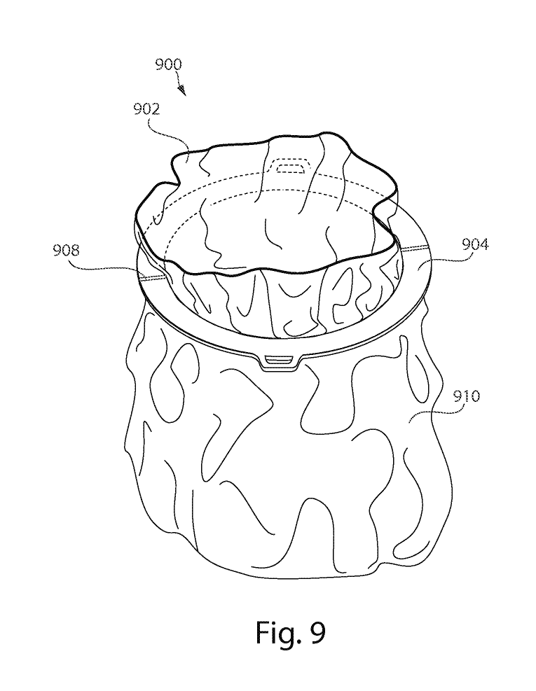

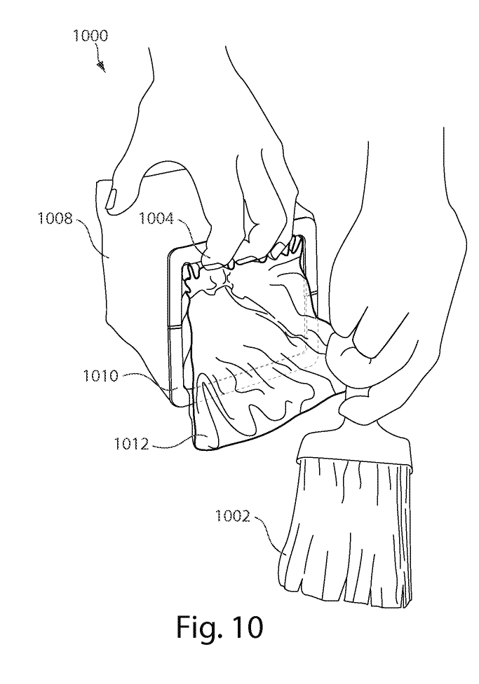

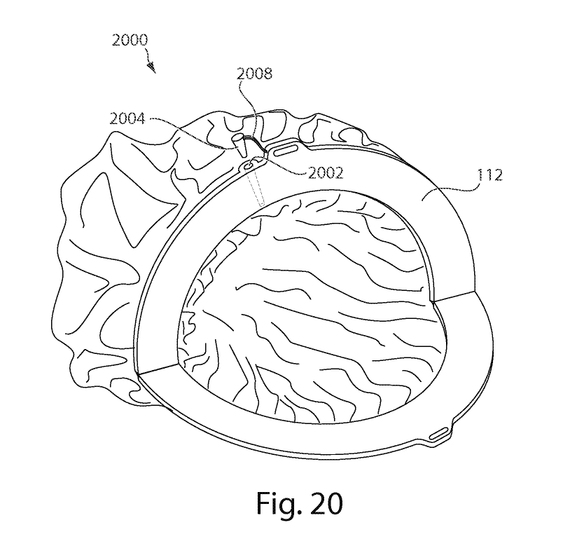

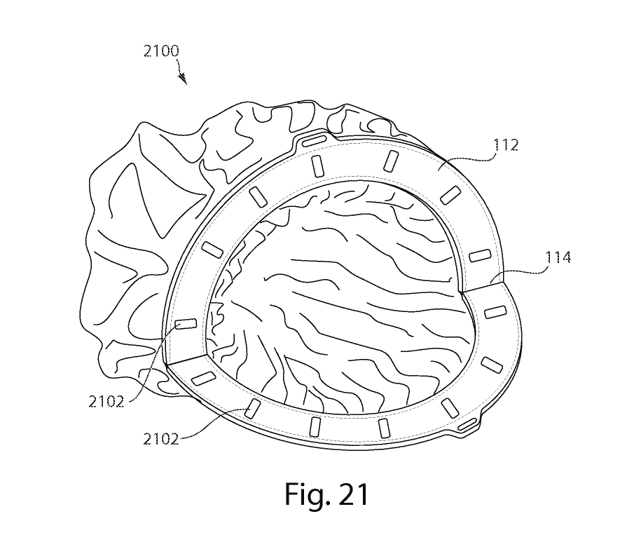

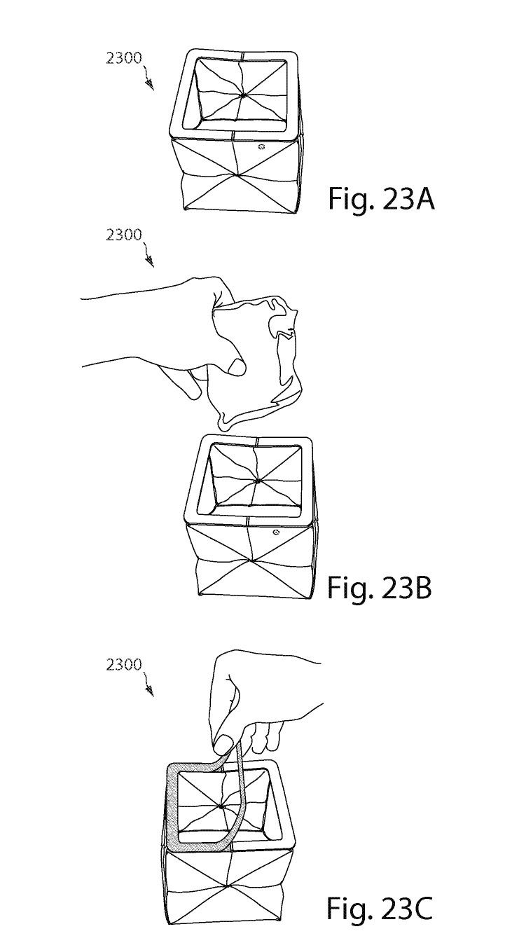

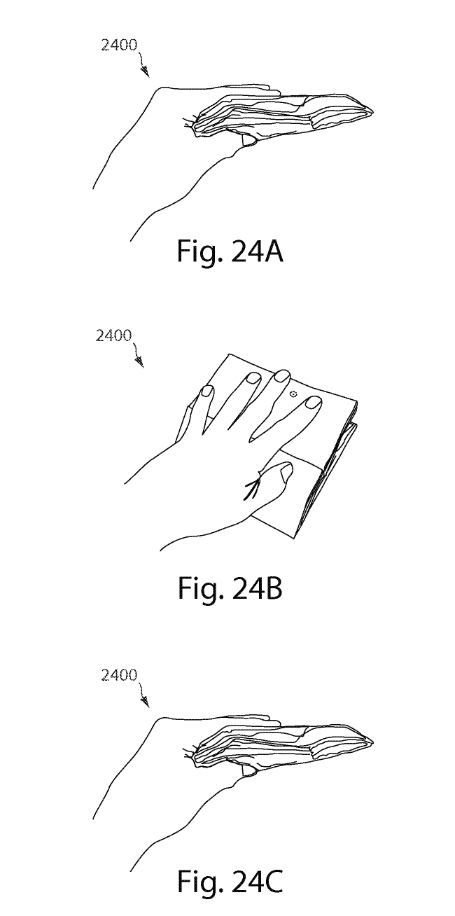

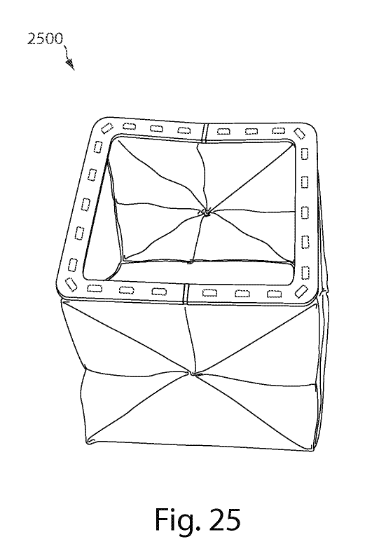

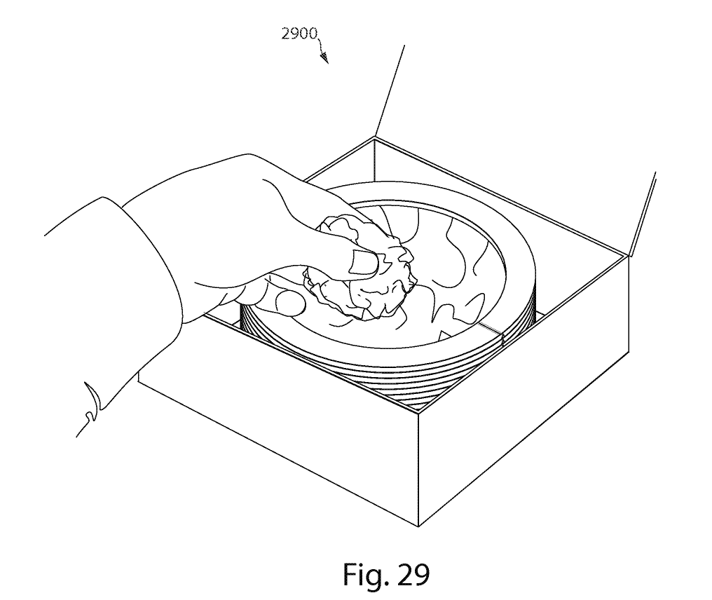

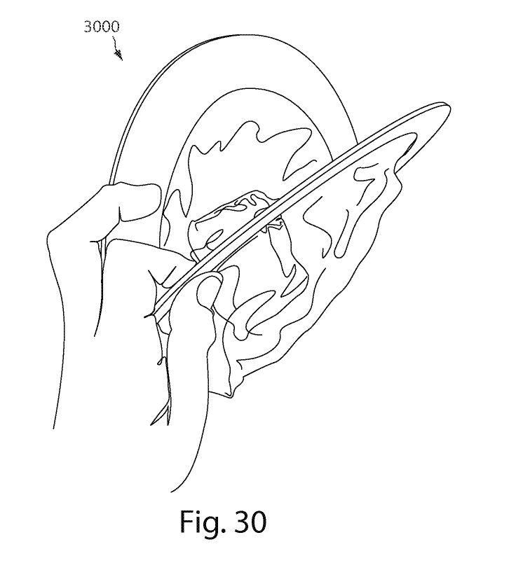

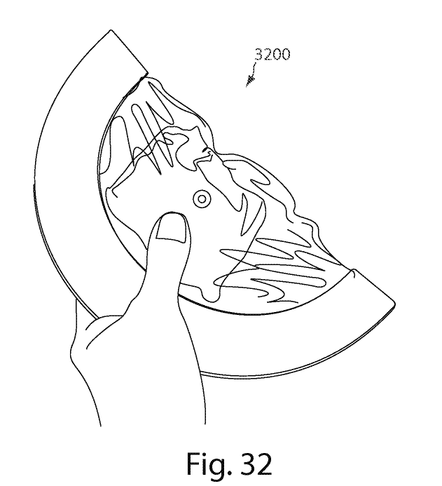

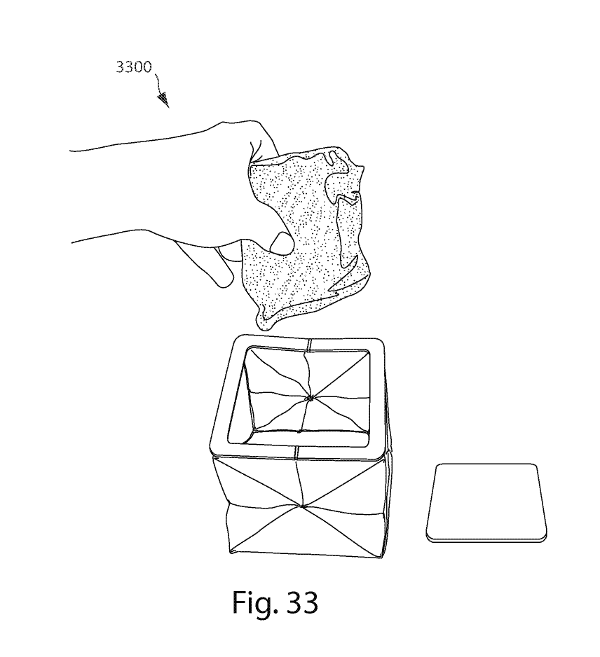

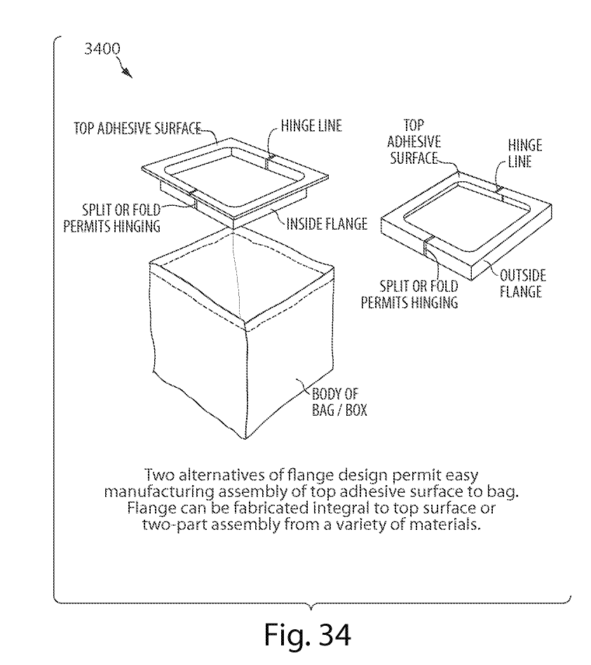

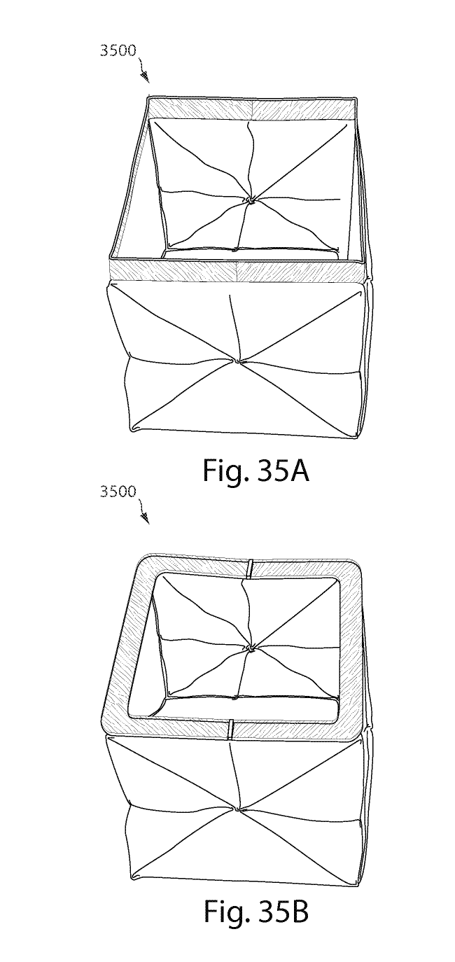

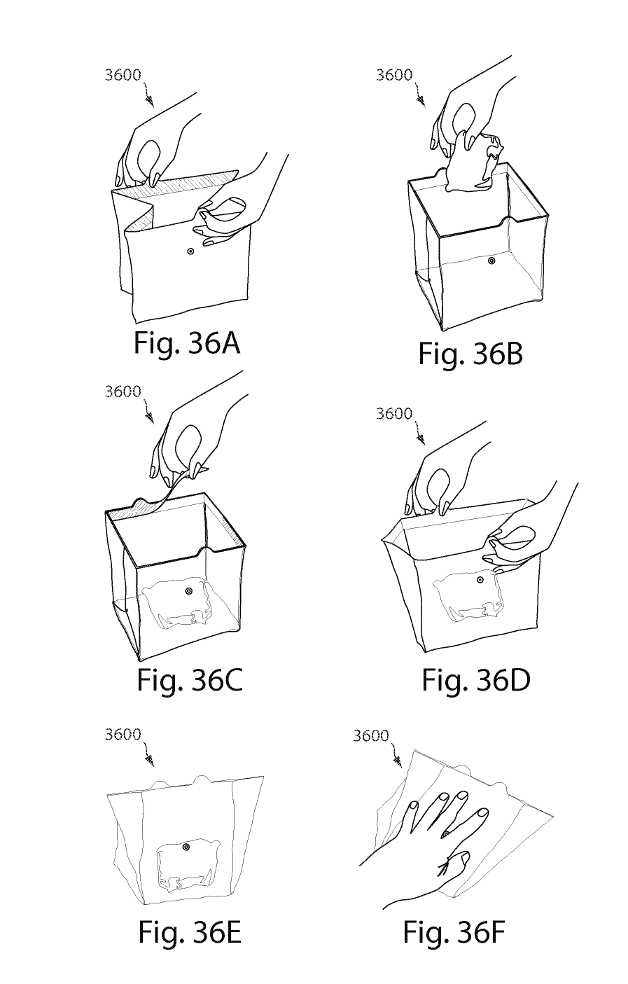

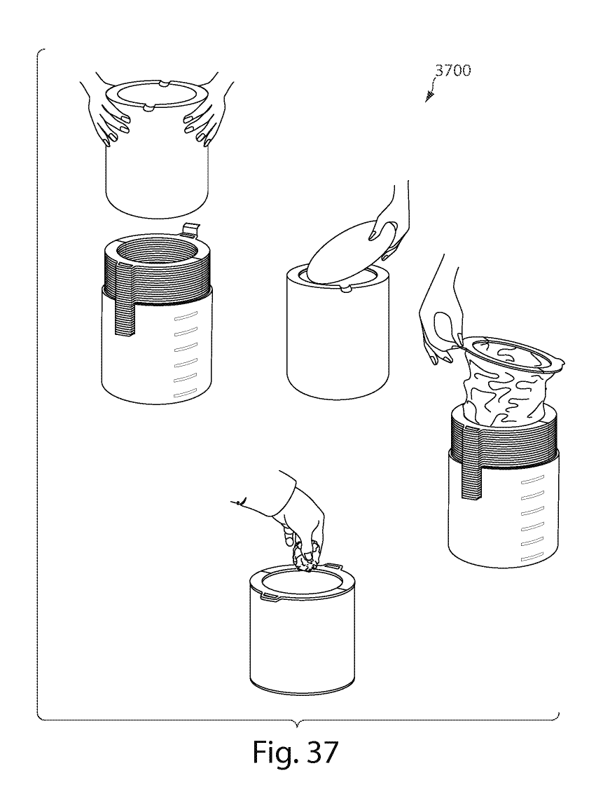

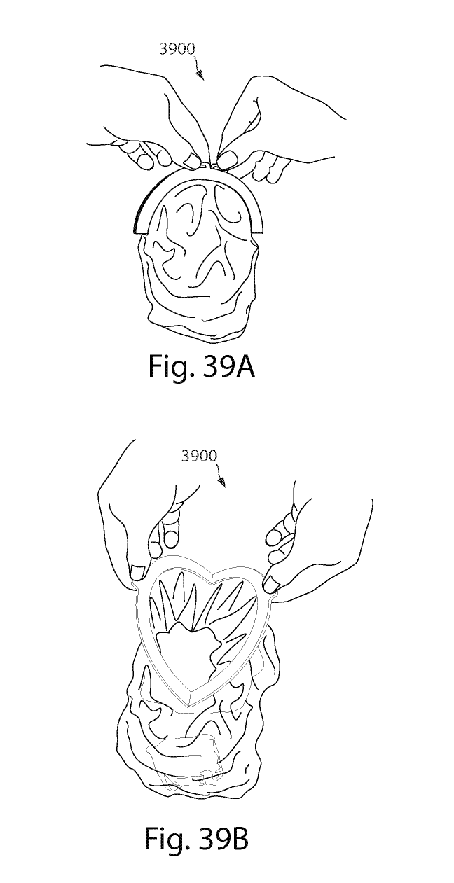

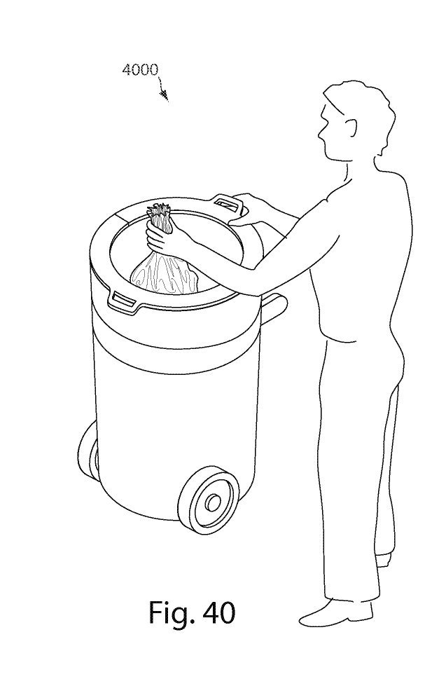

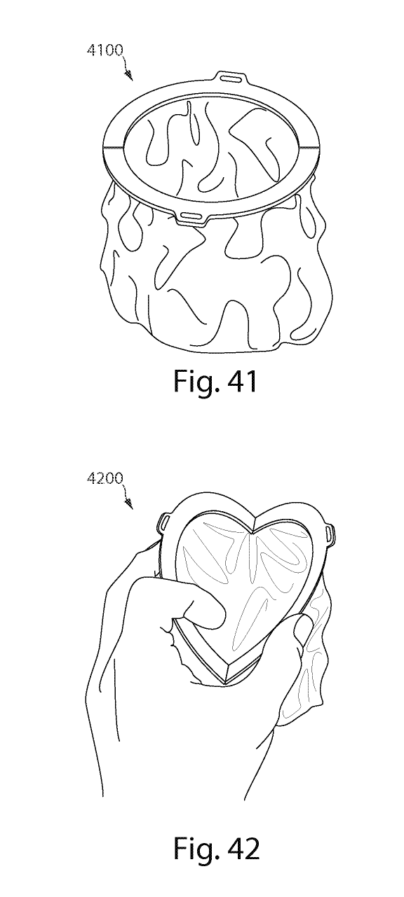

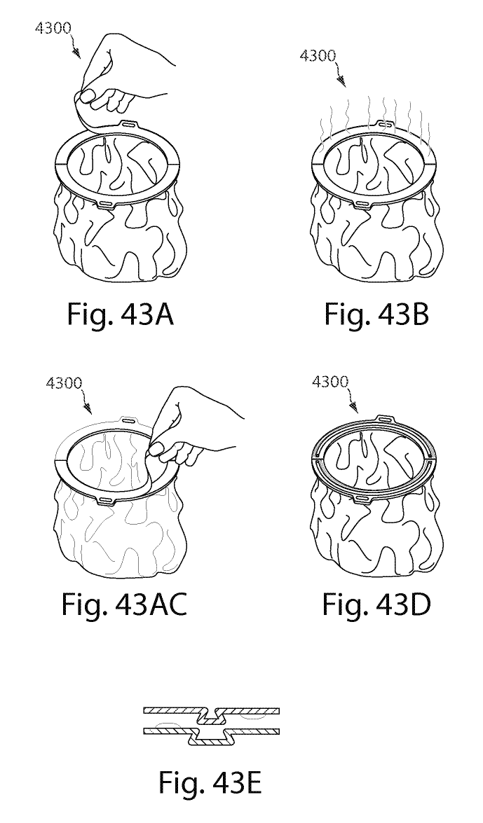

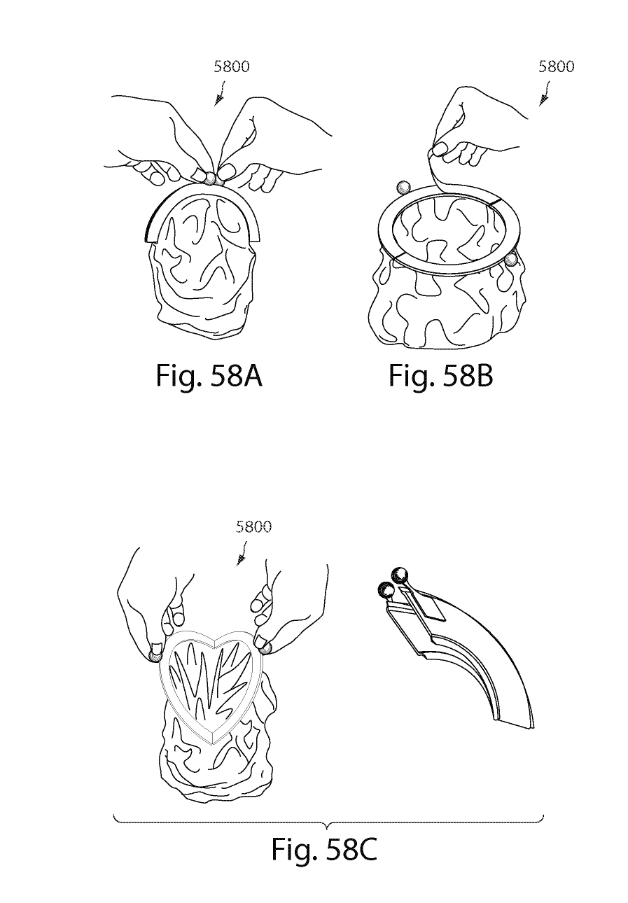

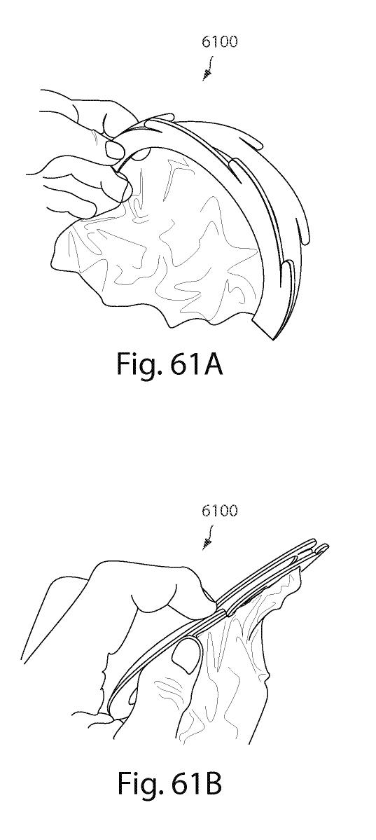

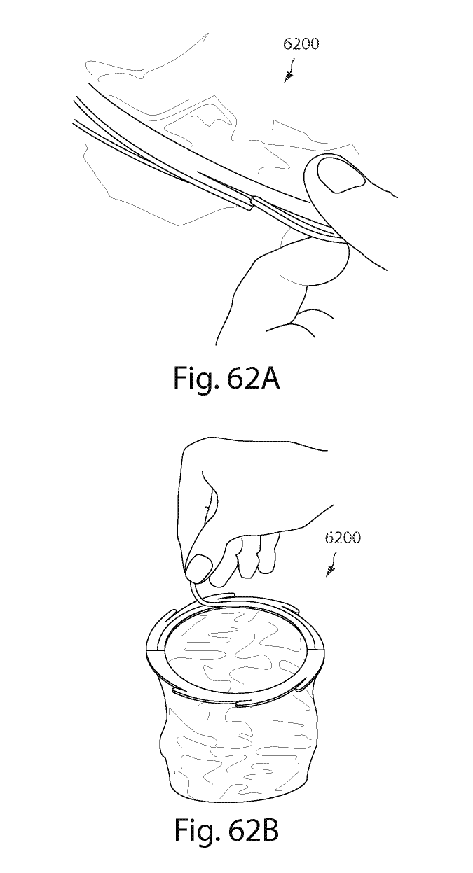

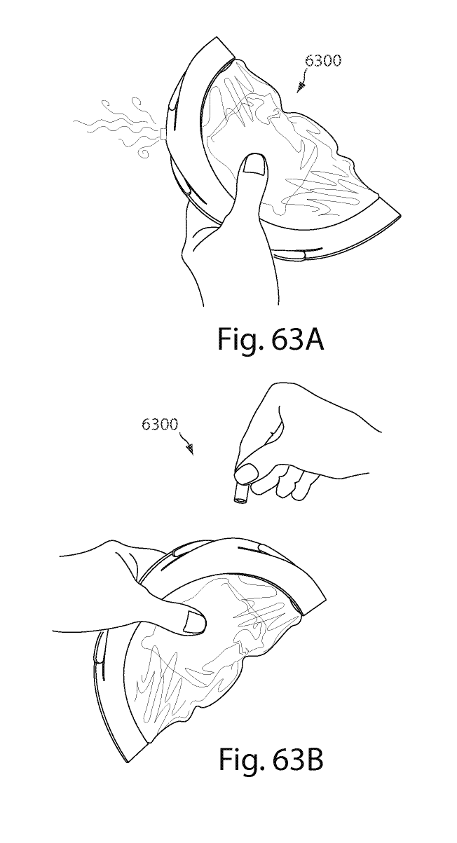

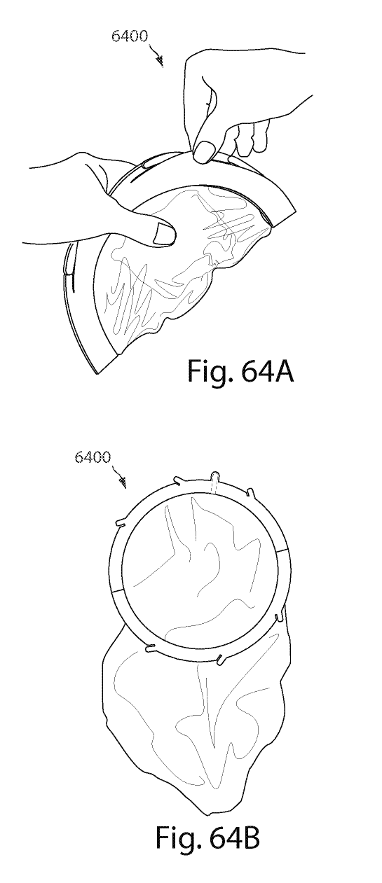

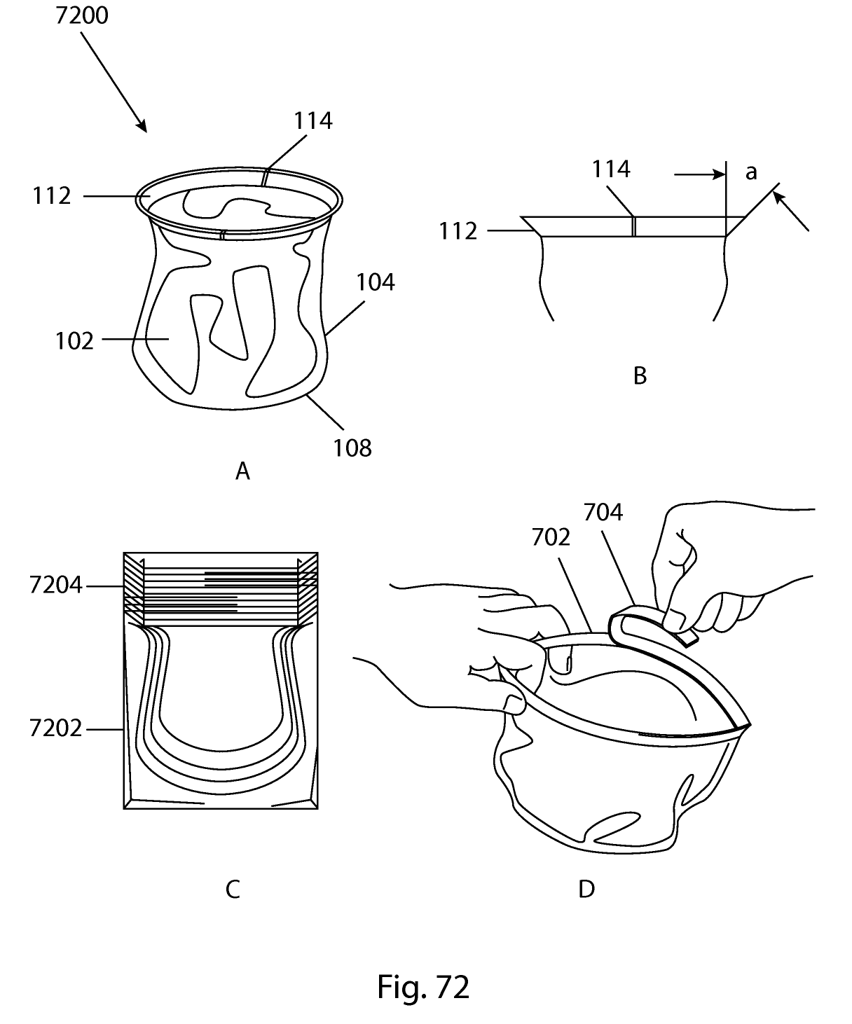

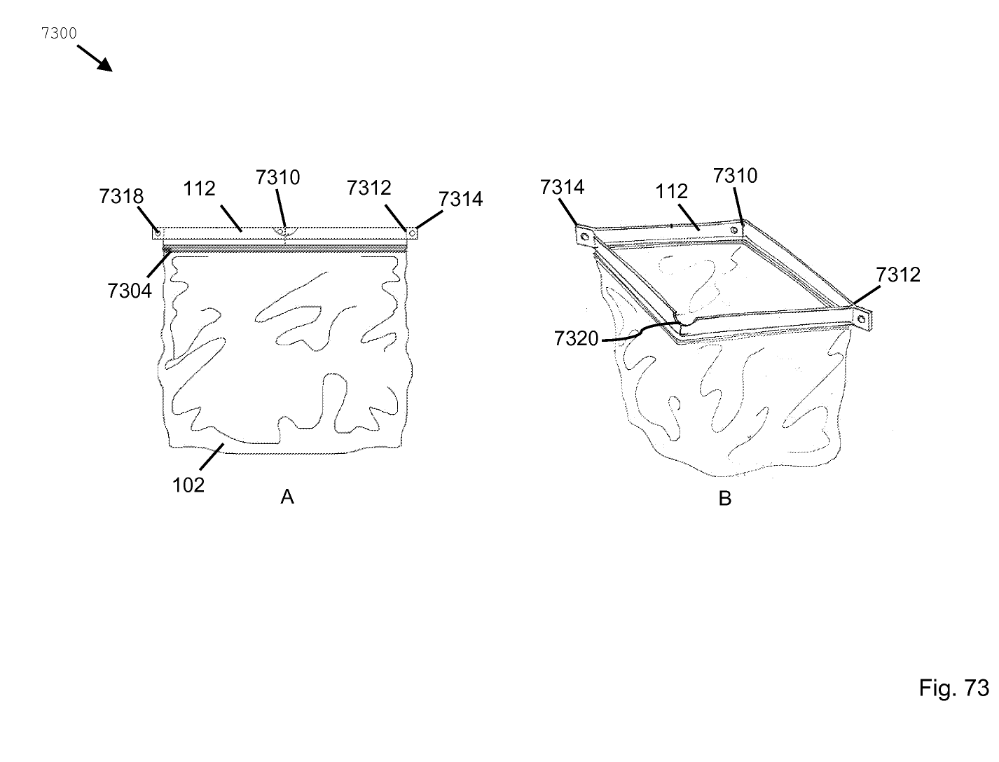

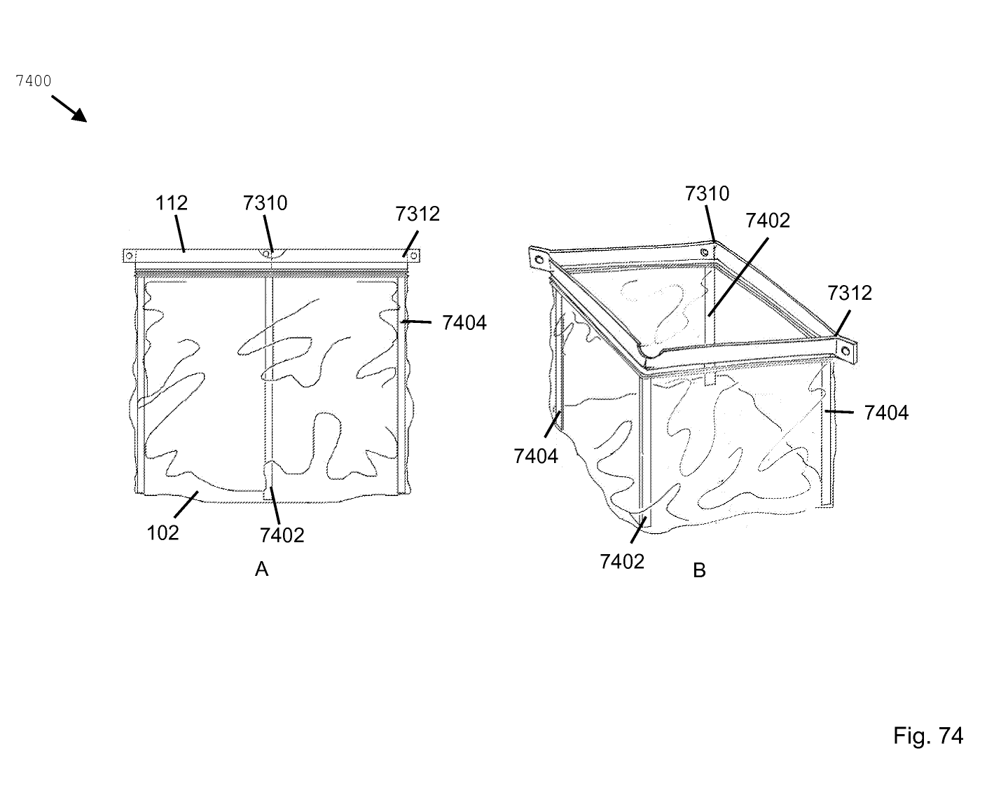

Referring to FIG. 1, an aspect 100 of the invention may involve a disposal bag with a frame. In an embodiment, the disposal bag 100 comprises a flexible material 102. The flexible material 102 may be plastic, polymers, paper, coated paper, fabric, a combination thereof, and the like. The plastic may comprise high density polyethylene, low density polyethylene, polypropylene, nylon, polylactic acid, a combination thereof, and the like. The flexible material 102 may be of any suitable gauge or thickness. The flexible material 102 may be biodegradable, chemical-resistant, waterproof, flame retardant, radioactivity-resistant, light-resistant, decorative, imprintable, and the like. The flexible material 102 may be impermeable to fluids and gases, such as for example, by being naturally impermeable, by applying a thin impermeable or laminate sheet material to the surface of the paper or other permeable material, by applying a waterproofing treatment to the interior or the exterior of the material, and the like. The flexible material 102 may be unstructured, as in FIG. 3 (302), FIG. 6, FIG. 9, FIG. 20, FIG. 21, FIG. 30, FIG. 31, FIG. 38, FIG. 39, FIG. 41, FIG. 42, FIG. 43, FIG. 44, FIG. 58, FIG. 61, FIG. 62, FIG. 63, FIG. 64, and FIG. 72A, or structured. For example, the flexible material 102 may form a substantially cylindrical shape with an unstructured bottom, as in FIG. 3 and FIG. 72A. In another example, the flexible material 102 may form a box, as in FIG. 1, FIG. 7, FIG. 10, FIG. 23, FIG. 25, FIG. 26, FIG. 33, FIG. 34, FIG. 35. FIG. 36, FIG. 73, and FIG. 74. The side walls 104 of the box may be constructed from a flexible material 102, as described herein, that may sustain a crease and be compliant enough to bend on moderate radii and return to the original plane. The crease in the side walls 104 may fold inwards or outwards.

The box may collapse to a compact form by creasing and folding the flexible material 102. In an embodiment, the disposal bag may be free-standing. The material and thickness of the bag walls may in part result in the free-standing capability. The cutting and arrangement of the sides of the bags may in part result in the free-standing capability. For example, the bottom surface 108 of the bag may be flat permitting the bag to stand on a flat surface. In this example, the bag may form a box with side walls 104 such as those described herein, a substantially cylindrical structure with a flat bottom surface 108, and the like. The exact geometrical configuration of the bag is not limiting for the present invention. In an embodiment, the side walls 104 of the box may be pre-folded 110 to create a collapsing structure that, when folded flat, may conform to a square. Storage of the box may be facilitated by stacking or arranging the collapsed structures. For example, multiple folded squares may be placed alongside one another in a storage cube. Preparing the folded box for use may involve unfolding the box by straightening the folded sides to a substantially vertical configuration such that the bottom is substantially flat and the frame is substantially parallel to the bottom. The box may be used for disposal with or without a frame 112 as described herein.

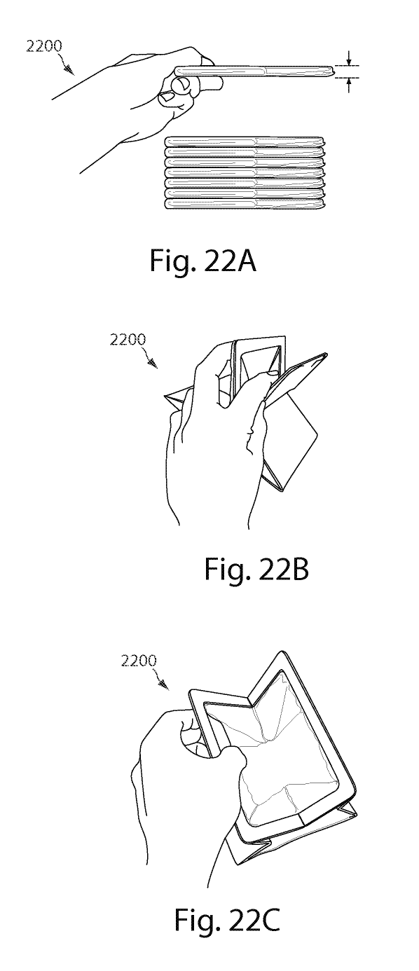

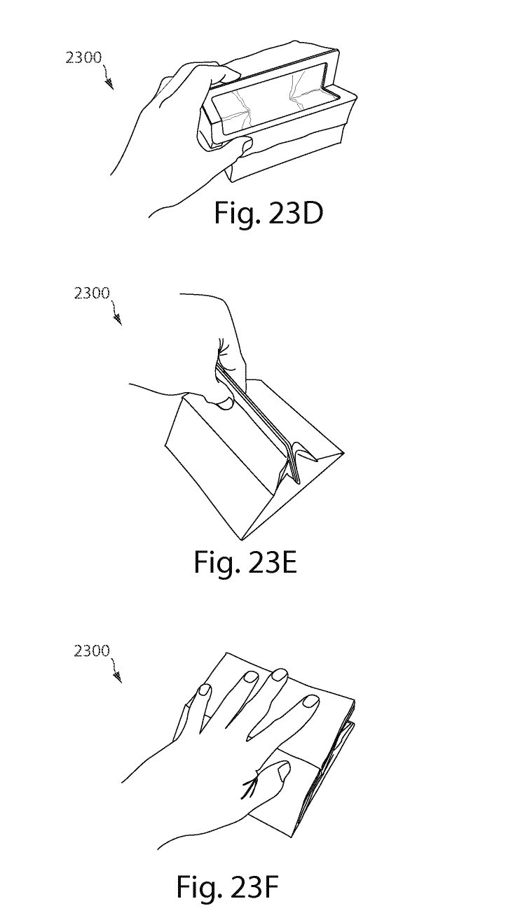

For example, FIGS. 22-24 depict use of a disposal bag with a substantially flat bottom. FIG. 22A shows the configuration of the disposal bag in its flat pack configuration. In FIG. 22B, the bag is opened by unfolding to reveal the opening in FIG. 22C. As shown in FIG. 23D, the bag may rest on the substantially flat bottom in a position that places the opening in a vertical configuration. Waste may be placed into the opening, as in FIG. 23E, and adhesive on the frame may be revealed by peeling off a protective layer, as in FIG. 23F. In FIGS. 23G & 23H, the process of sealing the mouth of the opening by folding the frame along a crease is depicted. The sealed bag may be further compressed for storage or ultimate disposal, as in FIG. 23I. The now compressed disposal bag, as in FIG. 24J, may be evacuated of excess air by compression and release of gas through an integral vent, as in FIG. 24K, to obtain a compressed, evacuated disposal bag, as in FIG. 24L.

In embodiments, the flexible material 102 may be configured into a cubical box-like or rectilinear structure, as in FIG. 73 and FIG. 74. For example, the disposal bag 100, made of the flexible material 102, may be designed in such a manner that it may be configured into a cubical box-like or rectilinear structure. The shape may be held by an attached frame, wherein the frame comprises a square or rectangular shape. In embodiments, as shown in FIG. 73, the disposal bag 100 may be unstructured and may not be free-standing. In another embodiment, the disposal bag 100, as shown in FIG. 74 may be free-standing because of the vertical legs 7402 and 7404.

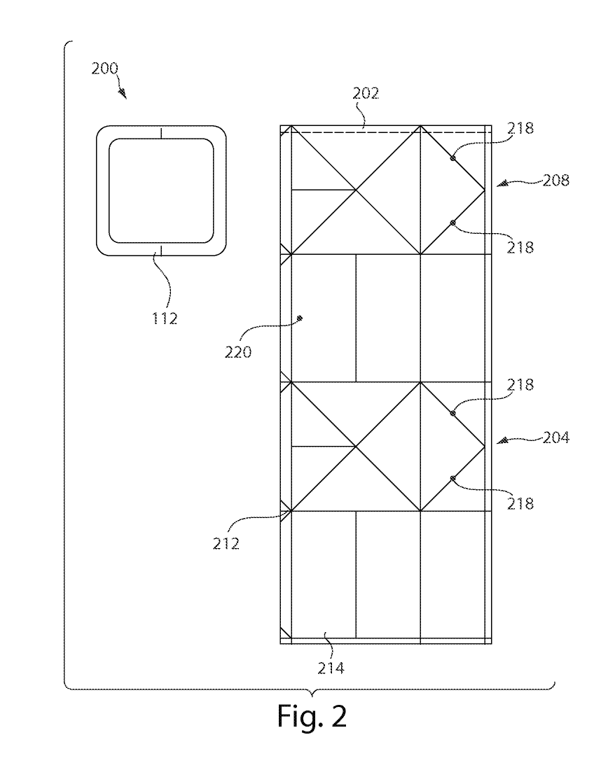

Referring to FIG. 2, in an embodiment, the box may be constructed in the manner of a standard flat bottom bag in its lower portion. The line pattern 214 may be creased to preserve the fold pattern once the flexible material is attached to the frame 112 in its three-dimensional form. The first lap joint 202 may create a tube from the flat sheet and may be sealed so as to be fluid and air impermeable. The bottom diagonal folds 218 may allow the flat bottom to be created as in other known flat bottom bag designs, first folding out in triangular double thickness ears on two opposing sides, creating the plane indicated with a heavy line from 204 to 208, with the central seam 210 overlapping by an amount that provides a water and airtight joint. Then, the ears may be folded under and attached to the square bottom surfaces. The top edge of the bag may be folded internally on all four sides with the corners double-folded over on each other on the diagonal lines 212 to form liquid/air tight corners. The third bonded seam may adhere the folded-in bag top to the perimeter frame in a fluid and air impermeable seal. Final folding on all of the other lines may compact the bag to a flat pack configuration. In this embodiment, the flat pattern for the box has a sealable valve 220 mounted in the material.

In an embodiment, the bag may be constructed from a continuous piece of material. Alternatively, the bag may be constructed from more than one piece of material. For example, the bag may be constructed from a single sheet of material that may have a cubic proportion. In another example, the bag is constructed from many pieces of material secured to one another through any conventional means. In an embodiment, the bag may be constructed using a sleeve-on-pipe manufacturing platform. The bag may be of any suitable size. In any event, the bag is constructed such that the material forms an opening in the bag.

In an embodiment, the bag may be clear, opaque, colored, patterned, and the like. The color may mark the contents within the disposal bag. The bag may include a written or graphical representation of the contents within the bag. For example, a marking of “Biohazard” with or without the recognized symbol for Biohazard may indicate that biohazardous content is contained within the disposal bag. In another example, the bag may be colored yellow and be marked “Radioactive Waste” with or without the recognized symbol for radioactive waste.

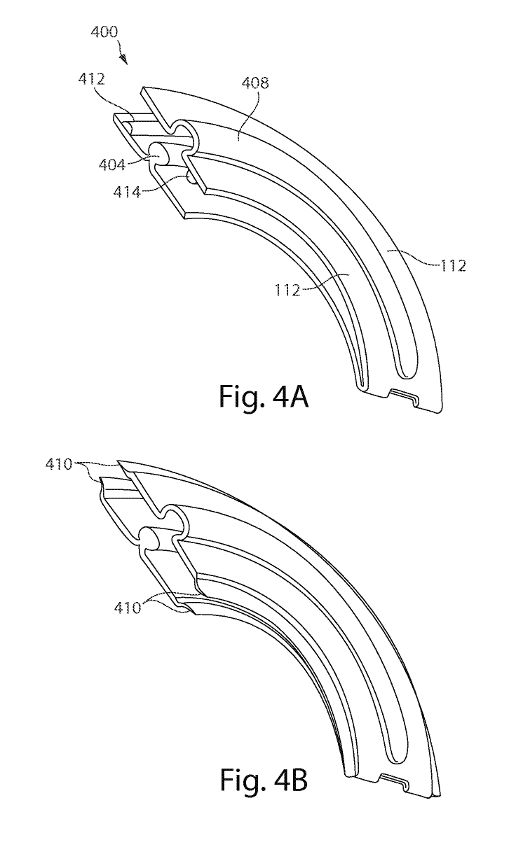

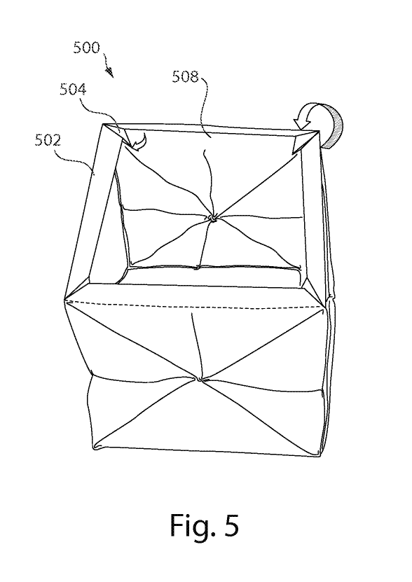

In an embodiment, the disposal bag may comprise a frame 112. The frame 112 may be associated with the bag at an opening in the bag. The frame 112 may be round, as in FIG. 3, round with a conical section, as in FIGS. 72A and 72B, oval, ellipsoidal, circular, square as in FIG. 1, rectangular as in FIG. 73, and FIG. 74, polygonal, molded, as in FIG. 4, and the like. The exact geometrical configuration of the frame is not limiting for the present invention. The frame 112 may be substantially flat. The frame 112 may be attached at an opening of the bag along the inner surface of the bag, along an outer surface of the bag, or a combination thereof. The frame 112 may comprise a flexible material, a rigid material, or a semi-rigid material, such as cardboard, plastic, or a planar material. The rigid or semi-rigid material of the frame 112 may enable the bag to be held in a substantially open configuration. The open configuration may enable hands-free use of the disposal bag by simply depositing the waste material into an open disposal bag and either sealing the bag immediately or sealing the bag at some time after the waste-making activity. For example, disposal of a soiled baby diaper into the disposal bag may not require removal of both hands from the baby. This feature may enable a user to dispose of waste without contamination of their hands.

In an embodiment, the frame 112 may be separately formed and attached to a bag by any method of attachment, including adhesive, thermal, heat sealing, physical, chemical, magnetic, ultrasonic welding, and the like. The frame 112 may be a single piece or formed from more than one part. The frame 112 may be molded from various polymer resins in a high-pressure injection molding process to include a suitable mechanical closure in the frame. Referring to FIG. 34, two alternatives of frame design facilitate manufacturing and assembly of adhesive surface to the bag. Both frames may be fabricated integral to the top surface or may be a two-part assembly of frame and bag. In one embodiment of the frame, there may be an inside flange to facilitate attachment of the frame to an inner portion of the bag. A split or fold in the frame may permit hinging. The top surface of the frame may comprise a sealing facility to permit sealing of the disposal bag. In another embodiment of the frame, there may be an outside flange to facilitate attachment of the frame to an outer portion of the bag. A split or fold in the frame may permit hinging. The top surface of the frame may comprise a sealing facility to permit sealing of the disposal bag.

The closure may comprise an interference locking system comprising one male and one female element that when pressed together may engage by mechanical spring interference to create an airtight seal. The closure may comprise a tongue 404 on one portion of the frame 112 that may move into engagement with a receiving cavity 408 on another portion of the frame 112 when a pressure may be applied to opposed sides of the frame once brought into close contact. The opening of the receiving cavity 408 may be of a smaller dimension than the tongue portion 404 and pressure may cause sufficient deformation at the entry to enable the tongue 404 to pass the opening lip and be fully enclosed by the cavity resulting in a tight mechanical engagement that may require significant force to disengage. An embodiment of such a sealing facility is also depicted in FIG. 43. The frame 112 may be supplemented by a gasket 412 or some other compressible or gap-filling material, as described herein. The gasket 412 may be disposed on the frame 112 such that it does not overlap with the interference locking system. For example, the gasket 412 may be disposed along an outer perimeter of the frame 112, along an inner perimeter of the frame 112, along an outer perimeter of one half of the frame 112 and along an inner perimeter of the opposing side of the frame 112, and the like. An alternative gasket solution may comprise a molded frame of diminishing dimension such that it may be relatively pliable at its outer surface 410 and may be formed to protrude toward the opposing feature 410 on an opposing side of the frame when in a closed configuration. When opposing sides of the frame may be brought into contact with one another, the compliant internal and external features 410 may deform one against the other creating an airtight and odor-proof seal along the perimeter of the frame 112.

In an embodiment, the frame 112 may be integrally formed with the bag such as by using a chemical or manufacturing technique that forms a heavier frame 112 out of a portion of the bag near its opening, such as in FIGS. 35 and 36. In an embodiment, the bag material may be extruded or otherwise formed from the heavier frame 112. For example, the technique may involve dipping the edge of the bag in a polymer liquid that may be heat or catalyst cured, a wax type material that may be applied and may be cooled, a paint type compound that may be air dried, a molding process such as insert injection molding wherein the frame 112 is formed to the edge of the material, and the like. There may be other materials and processes that may create a stiffness of the frame 112 and a smoothness of the frame surface that may facilitate the sealing function. For example, the top perimeter may be dipped or otherwise treated with a liquid or powder that may adhere to the edge inside and out, the application depth may be approximately that of the desired width of the frame. The liquid or powder may be capable of being formed, dried, and/or cured so that it may create a rigid surface that may incorporate the original material of the bag. Prior to forming, drying, and/or curing and while the material is still pliant, the material may be folded in any configuration so as to provide a frame 112 of suitable dimension. For example, a frame 502 may be formed by applying pressure on the inside and outside edge of the treated portion of the bag such that the material may be folded inward by a dimension desired for the width of the frame. The corners 504 may be folded to create an inward facing gusset that may be folded over upon itself so as to minimize the flat-folded dimension of the bag when stored or shipped. The corner joints may be filled in by chemical material or bag material so as to ensure a waterproof seal. A crease 508 may be formed as the frame 502 is created on the centerline creating a hinge point. In any event, the integral frame may comprise an adhesive for sealing the disposal bag, as in FIGS. 35 and 36, upon which may be disposed a peel-away protective layer.

In an embodiment, the frame 112 may comprise a handle 604. The handle 604 may be constructed as part of the frame 112. The handles 604 on opposing sides of the frame 112 may be offset one to the other or may overlap. The handles 604 may facilitate opening and closing the disposal bag. The handle 604 may comprise a hole. The hole in the handle 604 may facilitate hanging or otherwise temporarily suspending or storing the disposal bag and its contents between uses. The handle 604 may be located on only one side of the frame 112. The handle 604 may facilitate alignment of one or more frames 112 with one another and/or in a container.

In embodiments, the frame 112 may be round with a conical section, as shown in FIG. 72A and FIG. 72B. The frame 112 may have a semi-coplanar surface for adhering the disposal bag 100 to the frame 112 by methods known in the art. The semi-coplanar surface may be such that angle a is an acute angle. Because of the acute angle a, 90 degree deformation of the top edge of the disposal bag 100 may not be required for adhering the disposal bag 100 to the frame 112. In an embodiment, the frame 112 may be integrally formed with the disposal bag 100 such as by using a chemical or manufacturing technique that forms the frame 112 out of a portion of the bag near its opening. Further, top surface of the frame 112 may provide a wider surface for sealing the disposal bag 100 by methods described herein. The wider surface provided by the round frame 112 with a conical section ensures creation of an odor proof and air-tight seal.

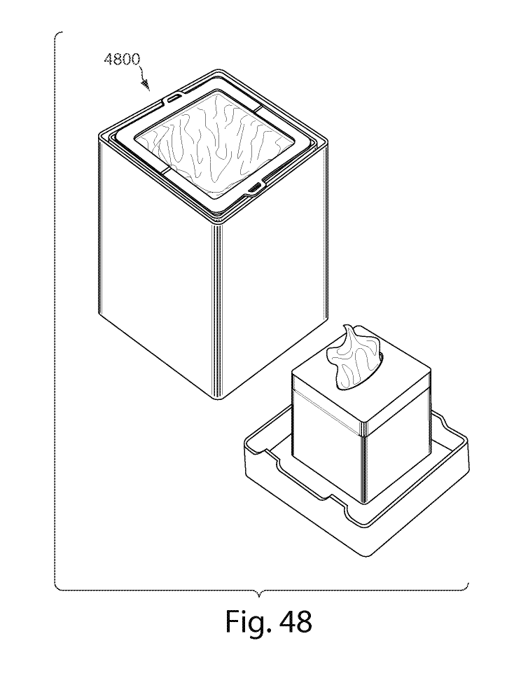

In an embodiment, the frame 112 may comprise a hinge 114. The hinge 114 may be formed in the frame 112 by a process that enables the rigid material to bend, such as by creasing, embossing, scoring, molding, perforating, stamping, cutting, breaking, and the like. The hinge 114 may extend partly or completely through the material of the frame. The hinge 114 may be located substantially at a centerline of a frame 112. The hinge 114 may be located at a bisecting point on a frame 112, also as in FIG. 41. The frame 112 may be able to fold at a hinge 114 such that one portion of the frame 112 may be brought in contact with another portion of the frame 112. Contact between opposing sides of the frame may be substantially uniform and airtight. When opposing sides of the frame are brought into contact with one another by pinching the hinge 114 or pulling up on opposing sides of the frame 112, the bag opening may be closed. The opposing sides of the frame 112 may be brought into contact with one another in a single-handed operation, as in FIG. 42 or FIG. 49, or with two hands. In an example, if the disposal bag is in the form of a box, the side walls 104 may follow the motion of the frame 112 when it may be folded to close the bag opening.

In embodiments, as depicted in FIGS. 73 and 74, the frame 112 may have hinge points at the middle 7310 of the frame 112 and at the end 7312. As described above, the hinge points 7310 and 7312 may be formed in the frame 112 by a process that enables the rigid material to bend, such as by creasing, embossing, scoring, molding, perforating, stamping, cutting, breaking, and the like. The hinge points at the middle 7310 may be located substantially at the centerline of the frame 112. Initially, the disposal bag 100 may be such that the hinge points 7310 are in contact with each other. As a result, the disposal bag 100 may have a substantially flat orientation. However, when the hinge points 7310 and 7312 are bent, such as to ninety degrees, the frame 112 may be able to configure to a square shape. This may, in turn, configure the disposal bag 100 into an approximately cubical or rectangular volume.

In embodiments, as depicted in FIGS. 73 and 74, the frame 112 may have a pair of tabs 7314. The pair of tabs 7314 may be extended ends of the frame 112 at the end hinge points 7312. The tabs 7314 may provide a means for packaging and handling the disposal bags 100 in a dispensing container. Further, each tab of the pair of tabs 7314 may have a hole 7318 in the middle of the tab. The hole 7318 may provide a registration feature for manipulating the tabs 7314.

In an embodiment, as depicted in FIG. 73, the disposal bag 100 may form a substantially cubical shape with an unstructured base. Because of the unstructured base, the disposal bag 100 may not have free-standing ability. However, in another embodiment, as depicted in FIG. 74, the disposal bag 100 may form a more structured cubical structure and may have free-standing capability. The free-standing capability may be because of vertical legs that may occupy the four corners of the disposal bag 100. The vertical legs may exist in pairs. For example, a pair of vertical legs 7402 may be attached at the middle of the frame 112 and a pair of vertical legs 7404 may be attached at the end of the frame 112.

In an embodiment, the frame 112 may comprise a sealing facility. Adhesive 702 may be used to form a temporary or permanent seal. For example, an adhesive 702 may be disposed along the surface of the frame 112. The adhesive 702 may be protected from accidental contact or contamination by a peel-away protective layer 704, a slippery interlayered separation sheet, by micro-encapsulation of the adhesive 702, by a two-part chemistry that is activated when adhesive layers on both sides of the frame 112 are brought into contact with one another, a combination thereof, and the like. For example, a peel-away protective layer 704 may be peeled away by grasping a portion of the layer not adherent to adhesive 702 and pulling, by physically separating the layer from the adhesive 702 to reveal a gap and continuing to extend that gap by pulling on the layer at the gap, and the like. A similar embodiment of an adhesive disposed on a disposal bag frame beneath a peel-away protective layer is depicted in FIG. 43. A peel-away protective layer 704 may not be necessary for micro-encapsulated adhesives or adhesives with two-part chemistry. After exposure of the adhesive 702, opposing sides of the frame 112 may be brought in contact with one another such that the adhesive 702 on at least one side of the frame 112 makes contact with the opposing side of the frame 112 to form a seal. A pressure may be applied to form the seal.

In an embodiment, the adhesive 702 may be disposed along the top edge of a bag where a frame may not otherwise be attached. For example, the bag may be in the form of a box wherein the side walls of the box may be creased and are able to fold inwards or outwards. As described herein, a low-tack adhesive 708 may be disposed along one side of the bag from a mid-point fold line on one side around to the mid-point fold line on the other side. The opposite side may be coated with a high-tack adhesive 702 between these fold lines. The box may be sealed, either temporarily or permanently, with the side walls folded inward or the side walls folded outward such that there may be a single crease at an outer edge of the adhesive band.

In an embodiment, the adhesive 702 may be low-tack, high-tack, and the like. For example, a low-tack adhesive 708 may provide a temporary seal. The bag may be re-opened after sealing the frame with a low-tack adhesive 708. When the bag is re-opened, the adhesive 708 may be available for resealing the frame. The low-tack adhesive 708 may continue to provide a resealable closure until the tackiness of the adhesive 708 may wear off. In another example, a high-tack adhesive 702 may provide a permanent seal. A high-tack adhesive 702 may be gap filling. For example, a gap filling permanent adhesive may fill the hinge joint when the frame is sealed.

In an embodiment, the adhesive 702 may be disposed on the surface of one side of the frame 112, both sides of the frame 112, a portion of either side of the frame 112, and the like. For example, the adhesive 702 disposed on one side of the frame 112 may be low-tack 708 and the adhesive disposed on the opposing side of the frame may be high-tack 702. In this example, exposure of a low-tack adhesive 708 may provide a resealable closure of the disposal bag until a permanent seal is desired. The low-tack adhesive 708 may provide a resealable closure against the protective film 704 covering the high-tack adhesive 702 on the opposing side of the frame 112. When a permanent seal may be desired, a high-tack adhesive 702 on the opposing side of the frame 112 may be exposed. A permanent seal may be formed when the high-tack adhesive layer 702 may be brought in contact with the low-tack adhesives 708 on the opposing side of the frame 112. The adhesion of the two different adhesives 702 to one another may fill the gaps of the sealed frame 112 and provide an airtight seal. For example, a portion of the adhesive 702 may be low-tack and a portion of the adhesive may be high-tack. A low-tack adhesive 708 may be disposed alongside a high-tack adhesive 702 on one or both sides of the frame 112. The adhesive layers may form stripes of adhesive 702 alongside one another. Multiple stripes of either adhesive 702 may be disposed along the surface of a frame 112. Each stripe of adhesive 702 may be protected as described herein. Exposure of a low-tack adhesive 708 may provide a resealable closure of the disposal bag until a permanent seal is desired. At that time, a high-tack adhesive 702 on the frame 112 may be exposed. A permanent seal may be formed when the high-tack adhesive layer 702 may be brought in contact with the opposing side of the frame 112.

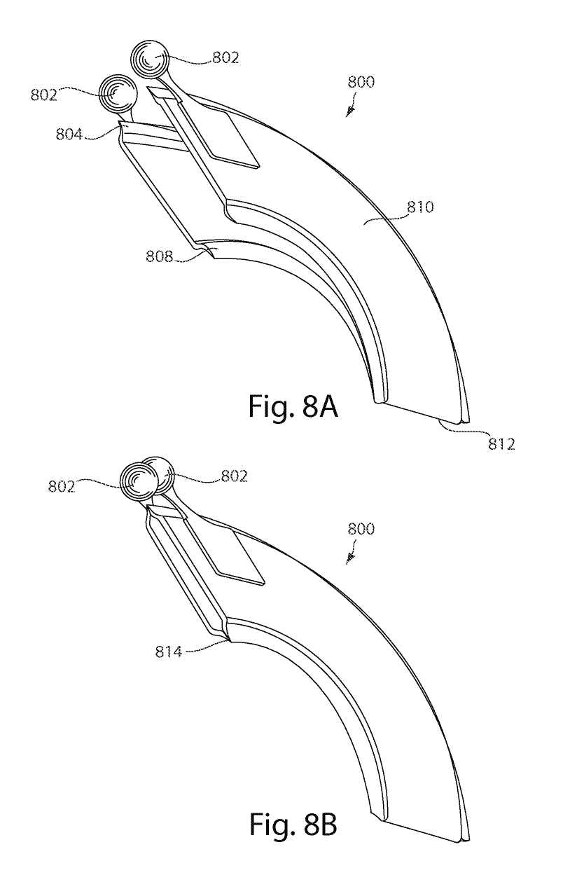

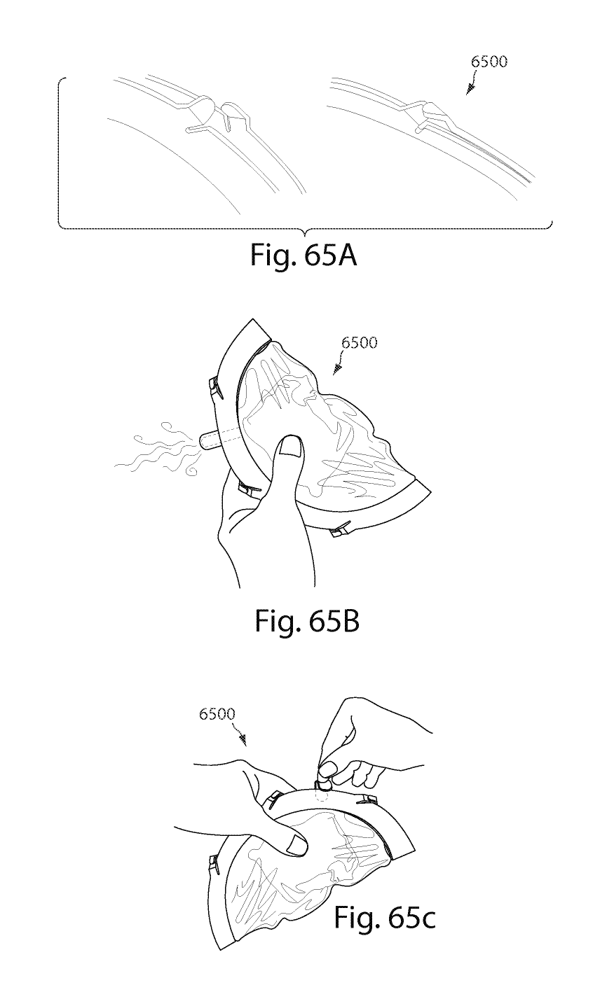

In an embodiment, the sealing facility may be mechanical. Referring to FIG. 3, FIG. 61, FIG. 62 and FIG. 65, the sealing facility may be a tab 304. The sealing facility may be a ball 802 or otherwise smooth geometric solid, as in FIG. 8 and FIG. 58. For example, a tab 304 or ball 802 on one side of the frame 112 may engage a tab 304 or ball 802 on the opposing side of the frame 112 when opposing sides of the frame 112 are brought into contact with one another to provide closure of the disposal bag. The opposed tabs 304 or balls 802 may deflect past one another such that when the deflected members may be past the centerline of both, they may engage such that their surfaces create a pressure between both that exerts a pressure on the two faces of the hinged top frame 112 keeping them in tight engagement. The closure of the disposal bag may be resealable by deflecting the tabs 304 or balls 802 past one another in the opposite direction to release the tabs' 304 or balls' 802 engagement. A single set or a plurality of opposed tabs 304 or balls 802 may be disposed anywhere along the perimeter of the frame 112. One or more sets of tabs 304 or balls 802 may be engaged to provide a resealable closure. In embodiments, the tabs 304 or balls 802 may facilitate handling of the disposal bag. The tabs 304 or balls 802 may be offset from one another such that opposed tabs 304 may not engage one another. In this example, the tabs 304 or balls 802 may not be intended for use as a sealing facility but may still facilitate handling of the disposal bag. A frame 112 with tabs 304 or balls 802 may be fabricated as a die- or laser-cut piece from flat stock. In an example, such as in FIG. 62, a frame with tabs may further comprise an adhesive layer with a protective cover. The frame may be resealable by deflecting the tabs past one another to open and close the bag. Once the user no longer wishes to open the bag, she may remove the protective layer from the adhesive and bring the two sides of the folding frame together. The adhesive may provide a permanent seal of the disposal bag, with or without additionally deflecting the tabs again.

In another example, the sealing facility may be a snap fit mechanism. The entire perimeter edge of the frame may comprise a zipper edge wherein the zipper edge of one side of the frame may compatibly engage the zipper edge of the opposing side of the frame to form a seal. In another example, the snap fit mechanism is a clip affixed to the edge of the frame. When opposing sides of the frame are brought into contact with one another, the clip may engage the opposing side of the frame to form a seal. In another example, the snap fit mechanism is a hook and eye. When opposing sides of the frame are brought into contact with one another, a hook affixed to the edge of one side of the frame may engage an eye that may be constructed as part of the frame. The eye may be located within the perimeter of the frame or may extend beyond the perimeter of the frame. In embodiments, the sealing facility may be a clasp as in FIG. 39 and FIG. 58, a zipper, a snap, a gasket, an O-ring type closure, a hook-and-loop, a spring-locking hinge, an electrostatic potential, a combination thereof, and the like. In another example, the mechanical closure may comprise discrete interlocks 2102, as in FIG. 21 and FIG. 25. In an embodiment, discrete interlocking features 2102 on opposing sides of the frame 112 may engage to form a mechanical seal. The interlocking feature 2102 may comprise a barb on one side of the frame 112 and a geometrically engaging receptor cavity on an opposing side of the frame 112. In another embodiment, the interlocking feature 2102 may comprise a ball on one side of the frame 112 and a detent on an opposing side of the frame 112. In another embodiment, the interlocking feature may comprise a snap hook on one side of the frame and a recessed edge on an opposing side of the frame. In another embodiment, the interlocking feature may comprise a snap on one side of the frame and a receiver on an opposing side of the frame. When the interlocking features engage 2102, a constant pressure may be applied on a gasket surface disposed along the frame 112 such that the gasket maintains an airtight seal. It may be understood by one of skill in the art that many different types of mechanical sealing facilities may be utilized in the present invention.

In an embodiment, the sealing facility may be magnetic (not shown). For example, a magnetically charged material may be disposed on one side of the frame 112 and a magnetically compatible material, such as iron, may be disposed on the opposing side of the frame 112. As opposing sides of the frame 112 approach one another, an attractive magnetic force may arise that may strengthen as opposing sides of the frame 112 are brought into closer contact to form a magnetically engaged configuration.

In an embodiment, the sealing facility may be thermal (not shown). A thermoplastic film may be disposed on at least one side of the frame 112. When opposing sides of the frame 112 are brought into contact with one another and a heat sealing tool is used to apply heat to the frame 112, the thermoplastic film may fuse to form a seal.

In an embodiment, there may be a plurality of sealing facilities on the frame 112. For example, a frame 112 may comprise tabs for a temporary seal and a high-tack adhesive may be disposed on the frame to provide a permanent seal. It may be understood by one of skill in the art that many different types of sealing facilities such as mechanical, molecular, chemical, electrical, electromagnetic, nanofiber, nanostructure-based connections, and the like may be used in the present invention. In any event, either a temporary or a permanent seal formed by any sealing facility described herein may be an airtight seal.

In embodiments, the sealing facility may be disposed on the disposal bag 100, as shown in FIG. 73 (7304).

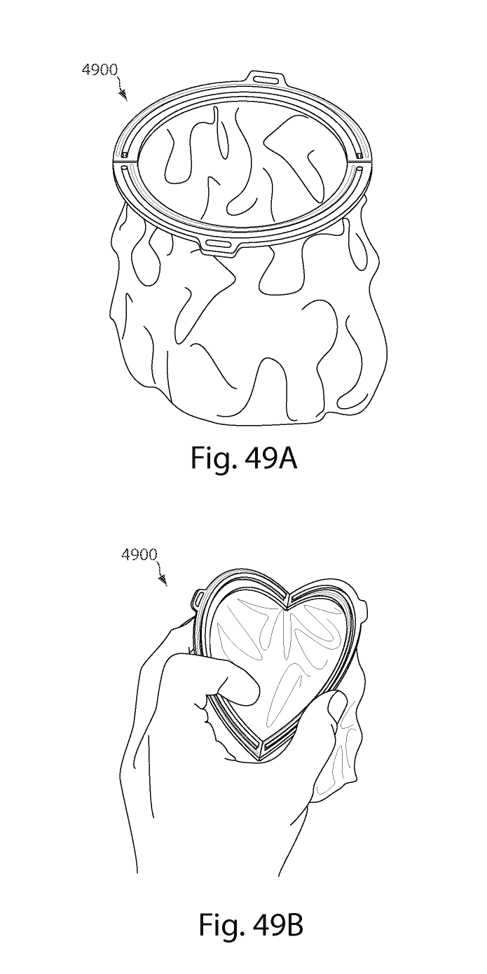

In an embodiment, a disposal bag may comprise a release facility. The disposal bag may be vented by applying a pressure to a sealed disposal bag to expel residual air through a release facility. Preventing the expelled air from reentering the disposal bag enables the waste to occupy a minimal volume for storage until ultimate disposal of the disposal bag. The release facility may comprise a vent tube 312 attached to the frame 112. The tube 312 may be straw-shaped as in FIG. 63 and FIG. 65, funnel-shaped as in FIG. 50, and the like. Upon sealing the frame 112, the tube 312 may remain lightly trapped in the frame 112. After sealing and venting the disposal bag, the tube 312 may be removed to allow an adhesive that may be adjacent to the vent tube 312 to make contact and complete the seal. For example, in FIG. 63 and FIG. 65, sealing of the disposal bag is accomplished by deflecting tabs on opposite sides of a folding frame past one another. A removable straw, shown in greater detail in FIG. 64 along with foam tape disposed along half of the folding frame, may be trapped in the sealed frame and may be used to release excess air from the sealed disposal bag. Once the excess air has been evacuated, the straw may be removed. In the configuration with foam tape, as in FIG. 64, the foam tape may expand to fill in the void left by removal of the straw to ensure an airtight seal. Alternatively, the tube 312 may be plugged or sealed with a separate member. The tube 312 may be plugged or sealed with material that is part of the frame 112 or bag or separate from the frame 112 or bag. The material may be coated with an adhesive to facilitate a tight seal.

In an embodiment, the release facility may comprise a channel 2002, as in FIG. 20, in the surface of at least one side of the frame 112, formed by the joining of the opposing sides of the frame 112, formed in a gasket, and the like. After sealing and venting the disposal bag, the channel 2002 may be plugged 2004, sealed, self-sealed, and the like. The channel 2002 may be plugged 2004 or sealed with material that is part of the frame 112, gasket, or bag or separate from the frame 112, gasket, or bag. The material may be coated with an adhesive to facilitate a tight seal. The release facility may comprise a vent, valve or a hole 118. The vent, valve or hole 118 may be mounted anywhere in the bag, such as in a portion close to the bottom of the bag, a portion close to the frame 112, a portion of the bag midway between the frame 112 and the bottom, and the like. The vent, valve or hole 118 may be formed into the material that may comprise the bag. The vent, valve or hole 118 formed from the material comprising the bag may protrude from the bag, as in FIG. 50. The vent, valve or hole 118 may be longer than it is wide which may facilitate collapse of the channel as a result of normal atmospheric pressure being greater than within the evacuated bag and inhibition of the entry of air into an outside portion of the channel, as in FIG. 50. The valve 118 may be a one-way valve or a two-way valve. After sealing and venting the disposal bag, the vent, valve or hole 118 may be plugged, sealed, self-sealed, and the like. The vent, valve or hole 118 may be plugged or sealed with material that is part of the frame 112 or bag or separate from the frame 112 or bag. The material may be coated with an adhesive to facilitate a tight seal. In any event, the sealing material may further comprise a microencapsulated adhesive that is activated by a pressure applied during sealing. A porous element impregnated with a fragrance may be placed inside a disposal bag such that air may pass over the fragrant material as it is being vented, as in FIG. 50B. In embodiments, the release facility may have a minimum release pressure, in that a minimum differential between the pressure within and without the bag may be required for actuation to vent air from the bag. In embodiments, the release facility may be filled with a fluid substance that may form a seal except when the bag may be pressurized. In any event, a plug may be tethered to the frame, tethered to the bag, or may be a separate member. The plug may be permanently shaped or may be shaped from malleable materials that deform to fit the release facility.

In an embodiment, the disposal bag may comprise a gasket 310. The gasket 310 may facilitate formation of an airtight seal. The gasket 310 may be any compressible or gap-filling material that provides an airtight seal, such as foam, rubber, elastic, closed cell foam, solid soft elastomeric material, void filling glue, spring material, folded plastic or paper, and the like. The gasket 310 may be disposed on the surface of at least one side of a frame 112, on both sides of the frame 112, inside a sealing facility, outside a sealing facility, and the like. The gasket 310 may protrude just past the hinge 114 line such that when the frame 112 may be folded on a centerline, the gasket 310 may fill the small void that may form at the hinge 114 line. An adhesive may be disposed along the surface of the gasket 310. The adhesive may be low-tack, high-tack, microencapsulated, and the like. The adhesive may be protected by a peel away protective layer. An odor reducing agent may be disposed in the gasket. An anti-microbial agent may be disposed in the gasket. The gasket may be used in combination with any of the disposal bag embodiments described herein.

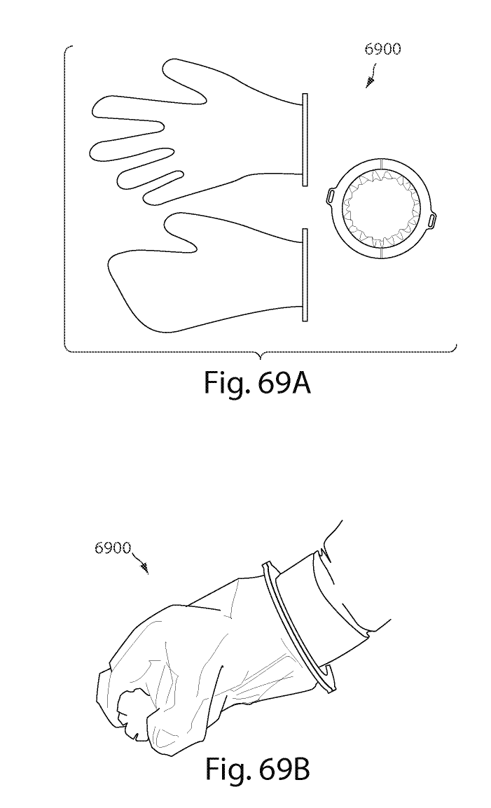

In an embodiment, the disposal bag may comprise an internal extension to the bag volume that may form a flap 902, as in FIG. 9, FIG. 38, and FIG. 69. The flap 902 may extend over a frame 904. The flap 902 may provide improved protection of the frame 904 from contamination during filling of the disposal bag. For example, an implement 1002 may be used to sweep material towards and into the bag without contaminating the frame 904. The flap 902 may be folded back into the bag prior to sealing the bag. The flap 902 may be attached to the frame 112 or may be integral to the bag.

In an embodiment, the disposal bag may comprise an odor reducing agent. The odor reducing agent may mitigate unpleasant odors associated with the disposed contents contained within the disposal bag. The odor reducing agent may provide a mood- or experience-enhancing effect, such as by providing a pleasant scent. The odor reducing agent may be a deodorant, odor neutralizer, air sanitizer, fragrance, scent, aroma, and the like. The odor reducing agent may be in the form of a liquid, an oil, a solid, a solid particle, a pellet, a capsule embedded in liquid, a powder, a talcum, and the like. The odor reducing agent may be disposed in one or more of an adhesive, a removable film, a frame, an exterior surface of the bag, an inner surface of the bag, a separate odor reducing member 3302, as in FIG. 33, an attached member, a waterproofing treatment, and the like. For example, a fragrance may be disposed in a foil-wrapped pouch that may be separate from the disposal bag. The odor reducing agent may be embedded. The odor reducing agent may be microencapsulated. The odor reducing agent may be activated by removal of a removable film. For example, removal of a removable film protecting an adhesive may expose a microencapsulated fragrance and the adhesive, as in FIG. 26 and FIG. 43. The fragrance may be released when pressure applied during the sealing of the frame breaks the encapsulation. Alternatively, a pressure may be directly applied to the adhesive to release a microencapsulated fragrance. The adhesive may be low-tack or high-tack.

In an embodiment, the disposal bag may comprise an anti-microbial agent. The anti-microbial agent may kill or inhibit the growth of microbes. The anti-microbial agent may prevent microbial contamination. The anti-microbial agent may be in the form of a liquid, an oil, a solid, a solid particle, a pellet, a capsule embedded in liquid, a powder, a talcum, and the like. The anti-microbial agent may be disposed in one or more of an adhesive, a removable film, a frame, an exterior surface of the bag, an inner surface of the bag, a separate member, an attached member, a waterproofing treatment, and the like. The anti-microbial agent may be embedded. The anti-microbial agent may be microencapsulated. The anti-microbial agent may be activated by removal of a removable film. For example, removal of a removable film protecting an adhesive may release a microencapsulated anti-microbial agent and expose the adhesive. The adhesive may be low-tack or high-tack.

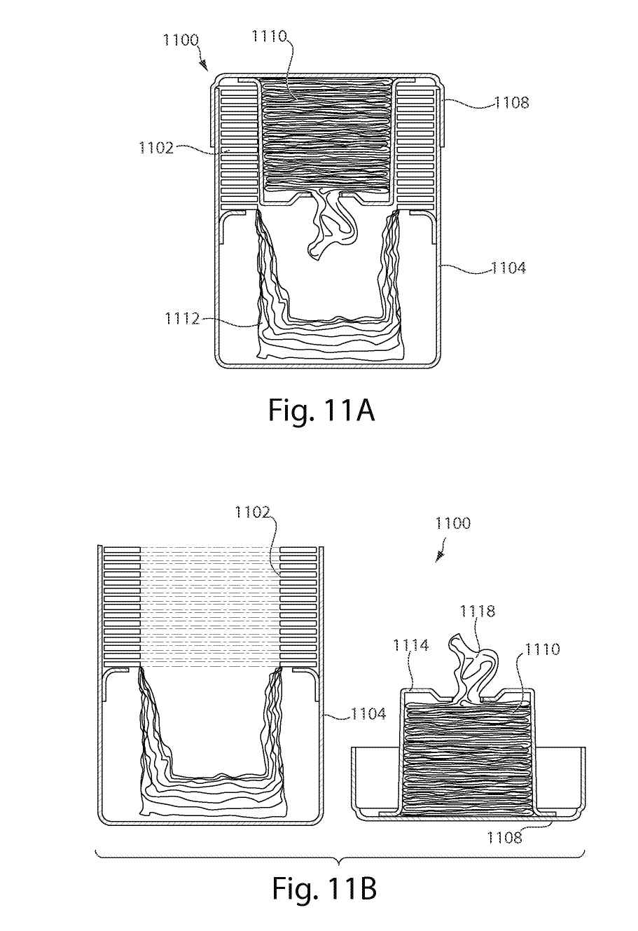

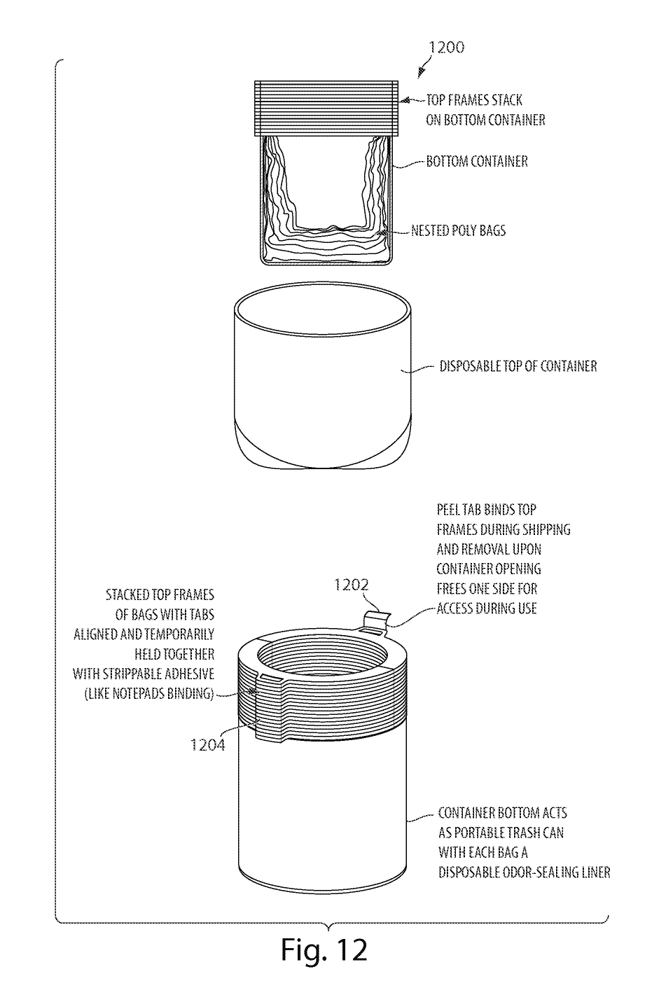

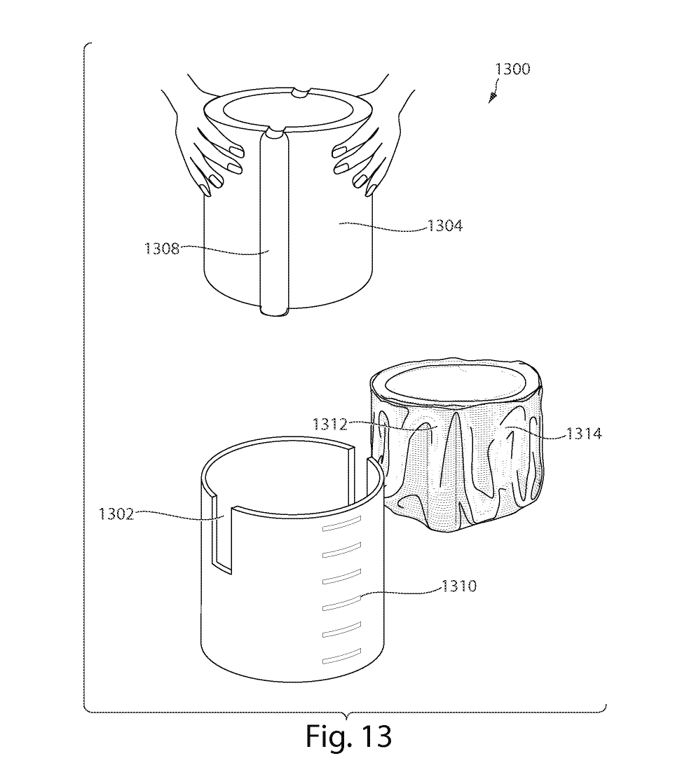

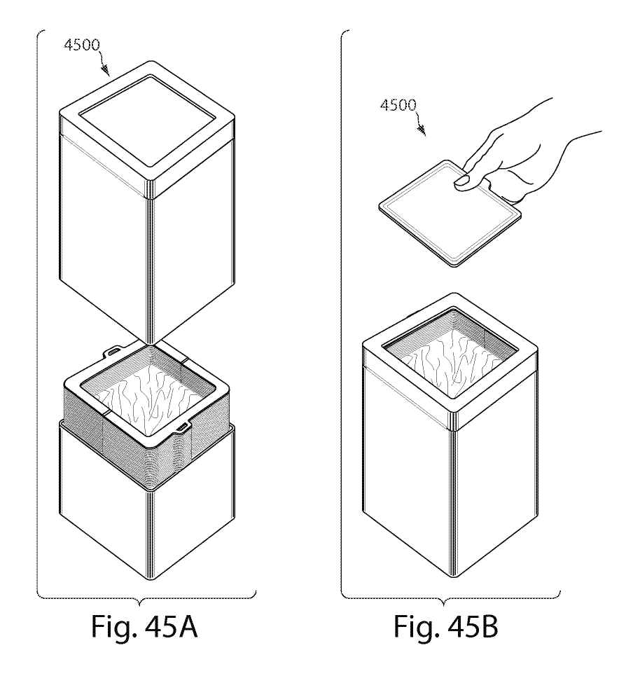

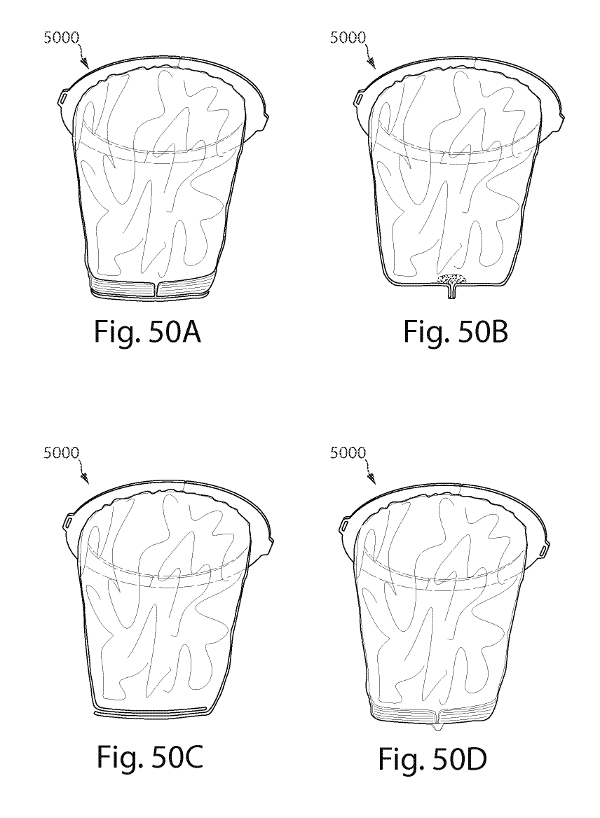

Referring now to FIG. 11, the frame 112 of the disposal bag may enable nesting 1102. For example, nesting may be where the frame of a first disposal bag may provide support on its upper surface for the frame of a second disposal bag when a second bag is positioned on top of the first bag such that the lower surface of the second bag's frame rests on the upper surface of the first bag's frame and the second bag extends into the first bag. A plurality of bags may be successively nested 1102 and the frames 112 may be successively stacked, one upon the other (see also FIG. 51). The stack of nested bags 1102 may be contained in a container 1104 for storing and dispensing the bags (see also FIG. 51). The container 1104 may facilitate a user using and/or pulling out one bag at a time. For example, waste may be placed into a top most nested bag 1102 (see also FIG. 46) while it is being supported by the stacked frames beneath it. In an embodiment, a top most nested bag 1102 may protect the frame 112 of a nested bag 1102 situated below it such that a peel-away adhesive protective layer may not be required on each subsequently nested bag below a top most nested bag 1102. The top surface of the frame 112 may be coated with adhesive while the underside may be provided with a non-stick backing surface that may protect the adhesive of the bag frame 112 underneath but that may easily release the bag frame 112 above when a user may pick it up from a stack of nested bags 1102.

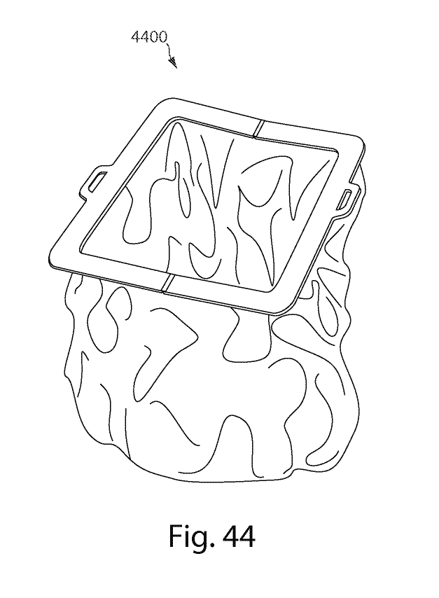

In an embodiment, the container 1104 may have an upper lip portion that may be flanged to support a frame 112 of a disposal bag. The container 1104 may be a cylinder, a canister, a box, a dispenser, a tub, a can, a bucket, and the like. The container may be made from plastic, cardboard, paper, polymers, metal, and the like. The container 1104 may be round, square or rectangular as in FIGS. 45-48, or any suitable shape. The container 1104 may act as a holder to support a bag while in use. The container 1104 may have a channel 1302 along an edge that may expose an aligned row of nested bag handles. The container 1104 may have a lid 1304. The lid 1304 may have a corresponding channel 1308 that may cover the aligned row of handles. The container 1104 may be sized to take up minimal space. The container 1104 may be sized to be easily located near a site of waste disposal, such as on a kitchen countertop, near a baby changing table, and the like. A stack of nested bags 1102 may be secured to one another and/or in the container 1104, such as for during shipping or storage. For example, a peel tab 1202 disposed along the vertical stack of bags 1204 at an outer perimeter of their frames may be used to secure the stack, such as during shipment. The peel tab 1202 may comprise a low-tack adhesive, a wax/plastic binding, or other substance that may allow the peel tab 1202 to be removed all at once or gradually as the stack of bags is used. In another example, a stripe of adhesive or wax/plastic binding may be disposed along the vertical stack without a peel tab 1202. In another example, the frames, tabs, or handles may have a slight interference feature that engages one to the next and holds the stack assembled until the individual selection and disengagement of the top most bag. In an embodiment, the container bottom may act as a portable trash can with each bag a disposable odor-sealing liner. It may be understood by one of skill in the art that many different types of stack securing methods, such as wax beads, plastic beads, and the like, may be utilized in the present invention.

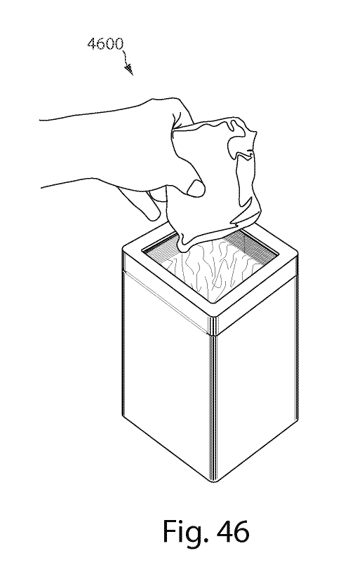

Referring to FIG. 45, the stack of nested bags may be concealed by a lid that covers the entire stack. The openings of the bags may be revealed by removing a portion of the lid, as in FIG. 45B, to expose the opening while protecting the frame. Waste may be disposed into the opening of the top-most nested bag, as in FIG. 46. Referring now to FIG. 47, a user may remove the top-most, waste containing disposal bag as in FIG. 47A, seal the bag as in FIG. 47B, and remove excess air as in FIG. 47C.

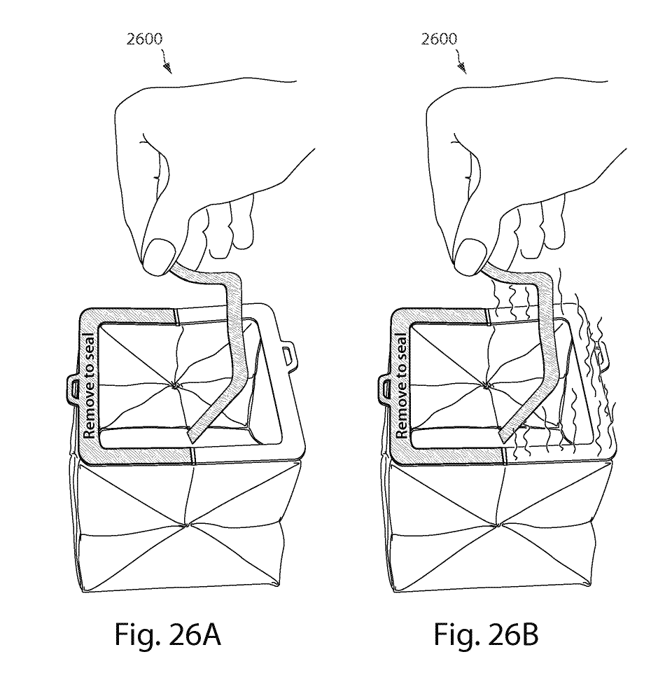

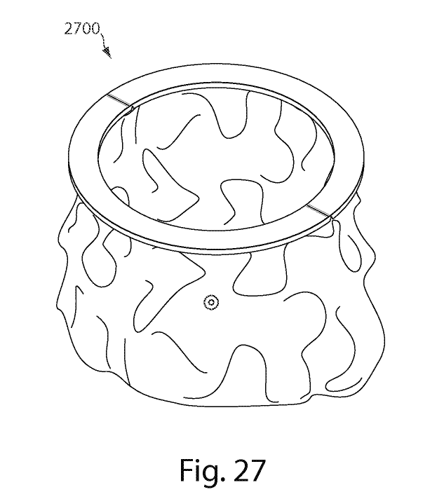

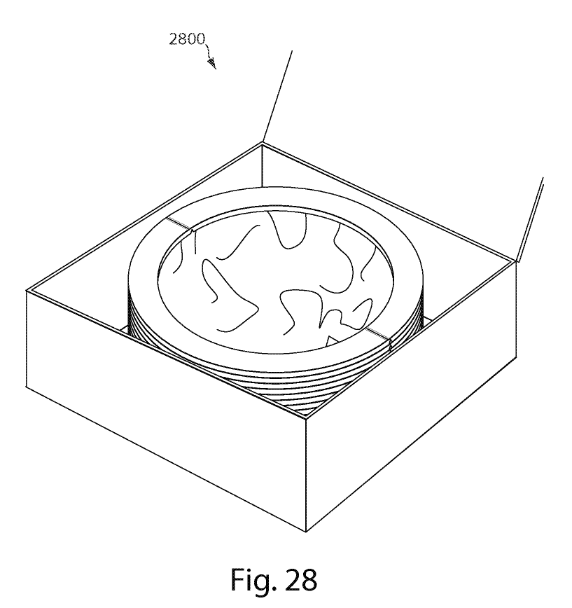

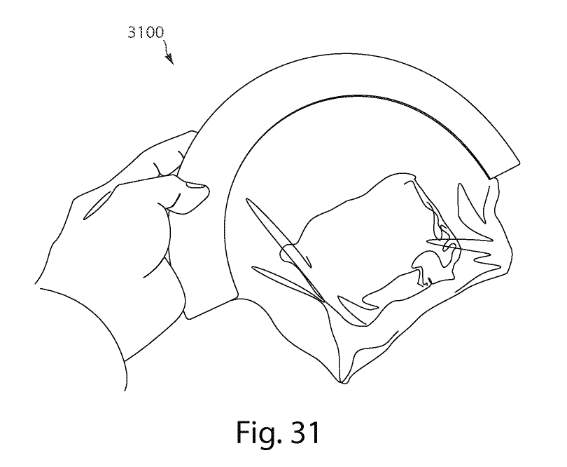

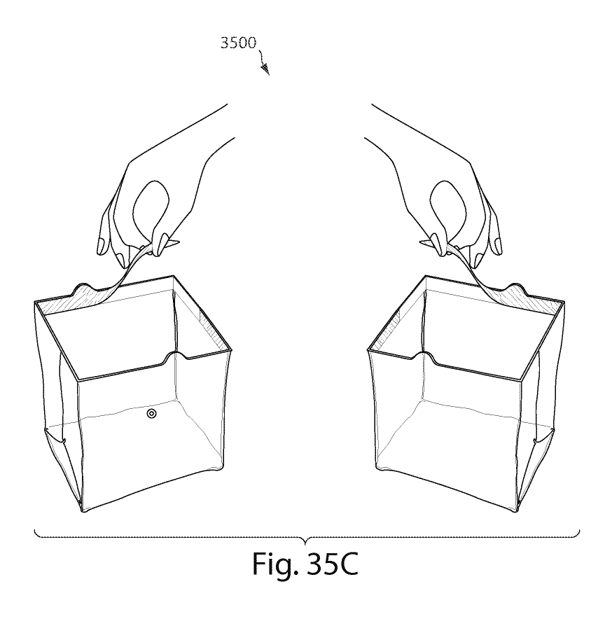

In another example, referring to FIGS. 27-32, operation of a nested disposal bag with a rounded frame is depicted. FIG. 27 shows the unstructured disposal bag with an integral release facility and rounded, hinged frame. In FIG. 28, a plurality of the bags shown in FIG. 27 are nested one on top of another in a dispensing container. Wate may be placed into the opening of the top-most bag, as in FIG. 29. Once the user decides to seal a waste-filled bag, they may remove it from the stack and fold the frame along the crease or hinge, as in FIG. 30, to seal the disposal bag with the contents contained therein, as in FIG. 31. The release facility may be used to remove excess air from the interior of the disposal bag, as in FIG. 32. FIG. 37 depicts another embodiment of use of the waste disposal system with a nested disposal bag contained within a dispenser.

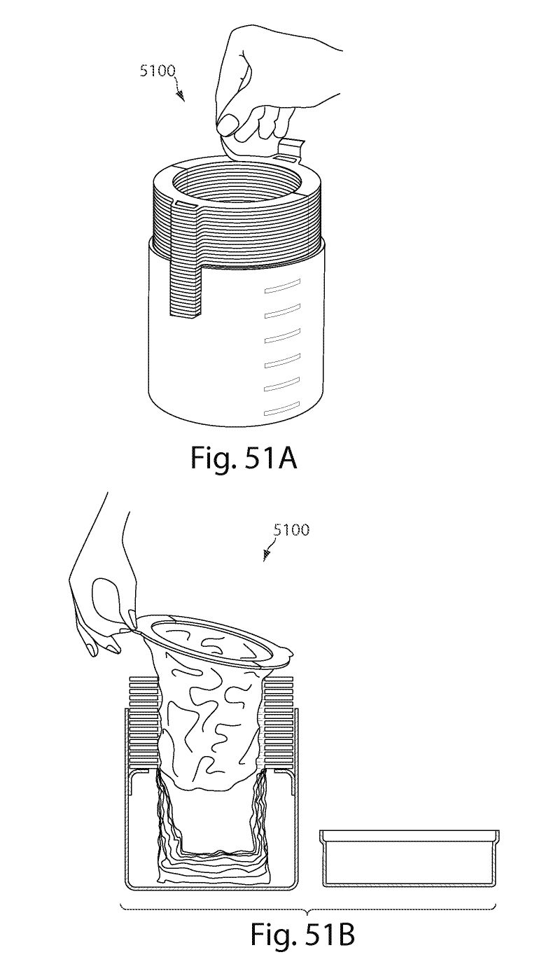

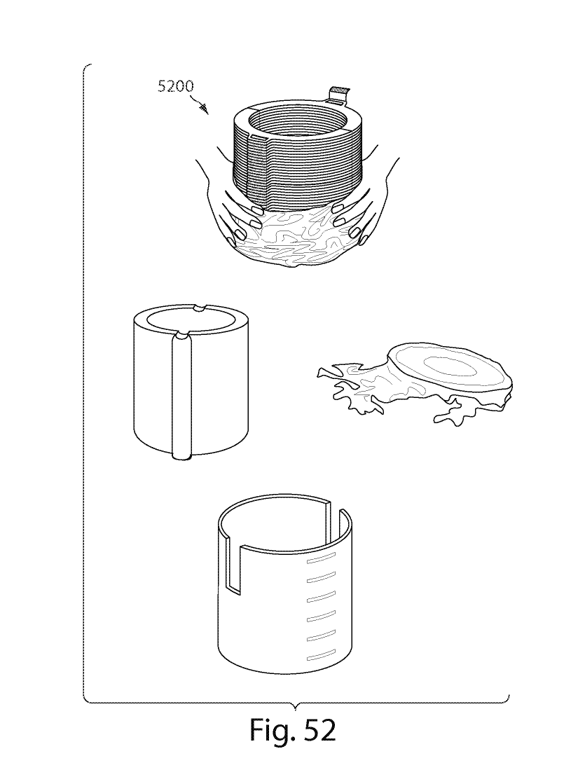

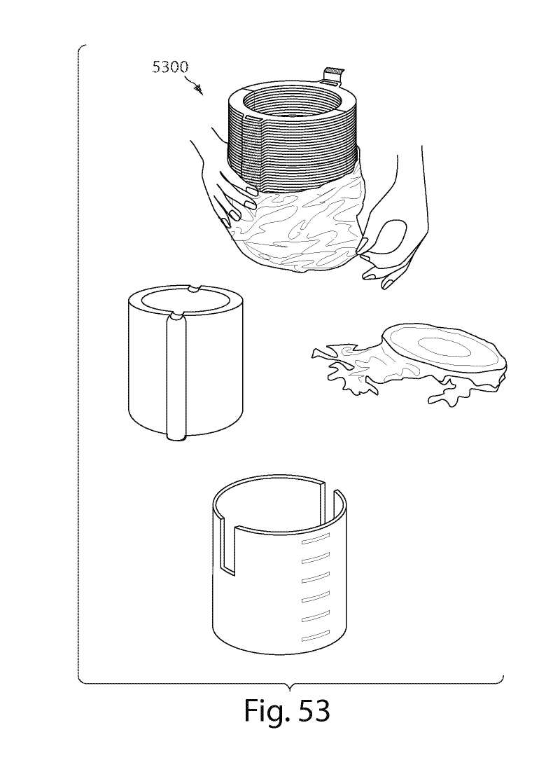

In an embodiment, once the initial supply of nested bags 1102 in the container is exhausted, the container 1104 may be refilled with a new stack of nested bags. The refill stack of nested bags may be contained in a refill pack 1314. The nested bags in the refill pack 1314 may be compressed for shipping and/or in-store display. The refill pack 1314 may be wrapped in a disposable container. The refill stack 1314 of nested bags may be secured to one another as described herein. FIGS. 52 through 54 depict another embodiment of a refill pack for a waste disposal system. In FIG. 52, the refill pack covering is discarded to reveal the refill pack. In FIG. 53, the disposal bags are extended to prepare for placement and use in a dispensing container. In FIG. 54, the refill pack is placed into the container where individual disposal bags may be dispensed from the top of the stack.

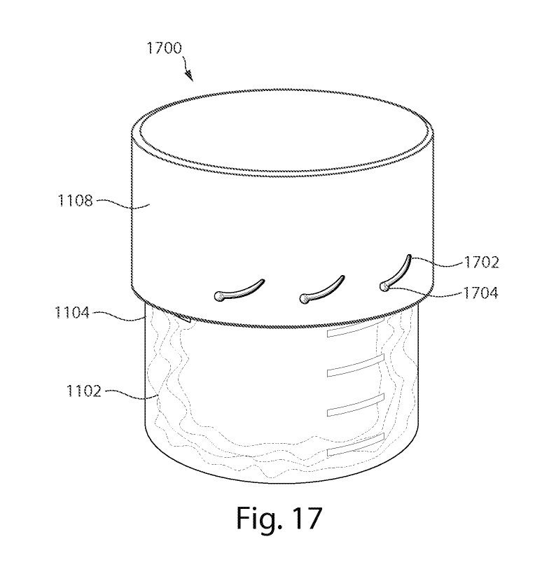

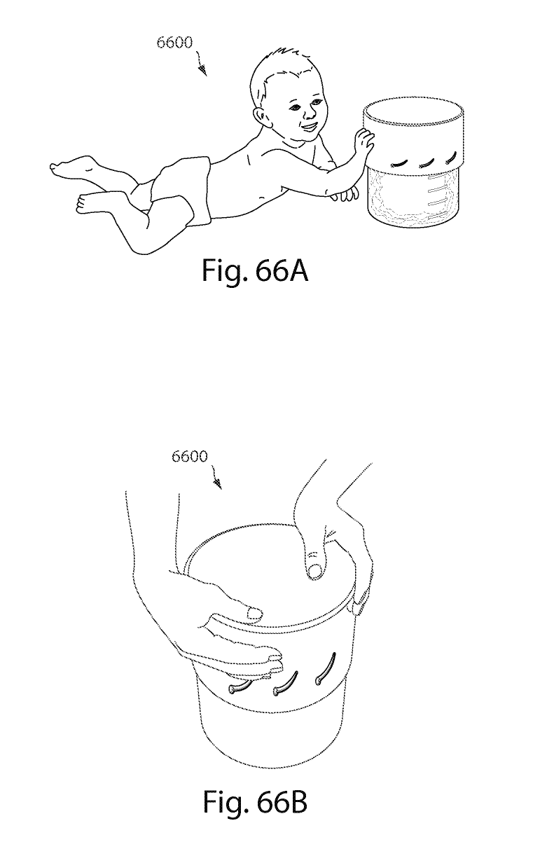

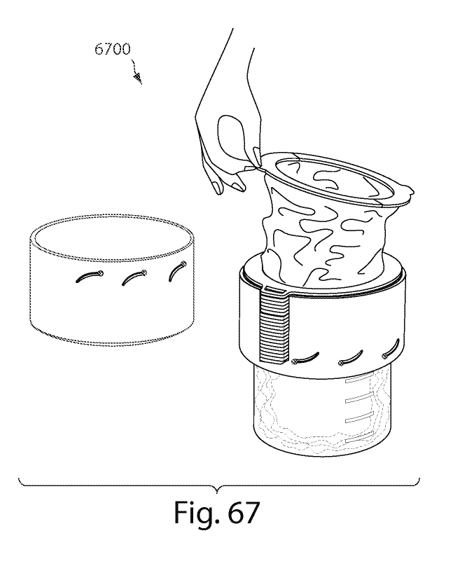

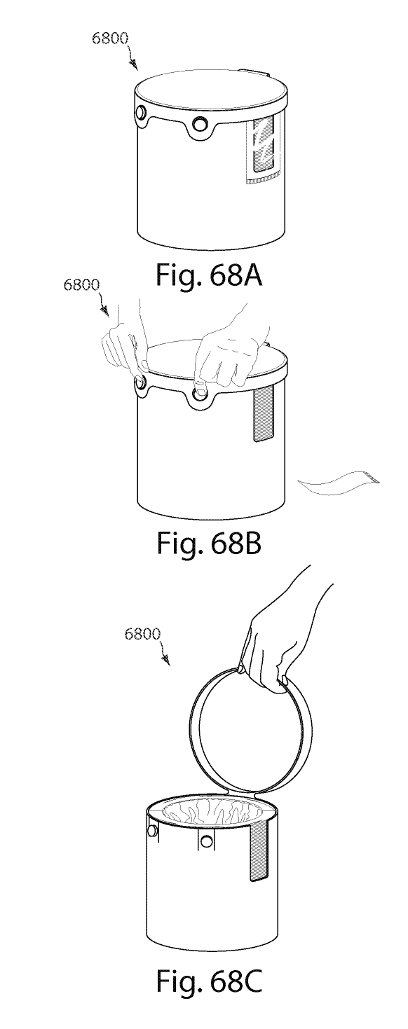

In an embodiment, the container 1104 may have a lower portion that substantially holds the nested bags and an upper portion that may act as a lid 1108. The lid 1108 may fit over a container 1104. The lid 1108 may have a lip that engages with a lip or indent on the container 1104. The attachment of the lid 1108 to the lower container 1104 may be accomplished by a close fit of the lid 1108 creating an outer sleeve fit over the inner similar profile of the container 1104. The lid 1108 may be attached to the container by a hinging mechanism. In an embodiment, the lid 1402 may swing open at a hinge 1404 to reveal the top most bag of a stack of nested bags 1408. The lid 1108 may have a locking feature. In an embodiment, the locking feature may be mating threads 1702, as in FIG. 17 and FIG. 66. Mating threads 1702 may be formed in both the lid 1108 and the base of the container 1104 so that when the lid 1108 is positioned in a sleeve fit over the upward extending side wall of the container 1104 and pushed into full engagement, the outward protruding threads 1702 of the container 1104 will intercept the inward protruding threads of the lid 1008 so that by twisting the lid 1108 relative to the container 1104, the lid 1108 may be drawn down into locked engagement with the container 1104. A further downward twisting force on the lid 1108 against the top edge of the container 1104 or against another designed spring feature within the top or bottom of the container 1104 may cause an interference engagement at the end of the thread. There may be a protrusion 1704 out of the line of the formed thread against a similar counter-protrusion that provides a locking feature so that without downward pressure the reverse twisting motion will not disengage the thread. In another embodiment, the locking feature may be a spring loaded snap lock engagement 1412. Spring loaded snap lock engagement features 1412 may be formed in the base of the container so that when the lid is closed in a sleeve fit over the upward extending side wall of the base, an opening in the side wall of the lid may accept the protruding snap lock button feature 1412 to lock the lid in the closed position. The deflection of the material of the side wall of the base may provide the spring pressure required to engage the snap lock feature or another discrete physical spring may be used. Opening the container may involve depressing a snap lock button 1412 formed into the base wall of the container through holes in the lid of the container, so that lifting the cover disengages the lid from the base of the container. Another embodiment of a snap lock engagement feature may be depicted in FIG. 68. In any event, the edges of the top frames of the nested bags may be exposed by an opening in the side wall of the dispensing container and prior to use this opening may be protected by a removable film or other device that inhibits contamination of the bags, as in FIG. 67.

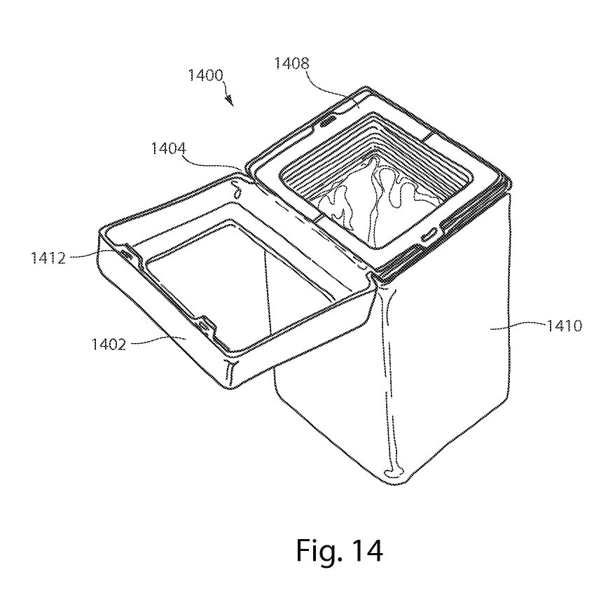

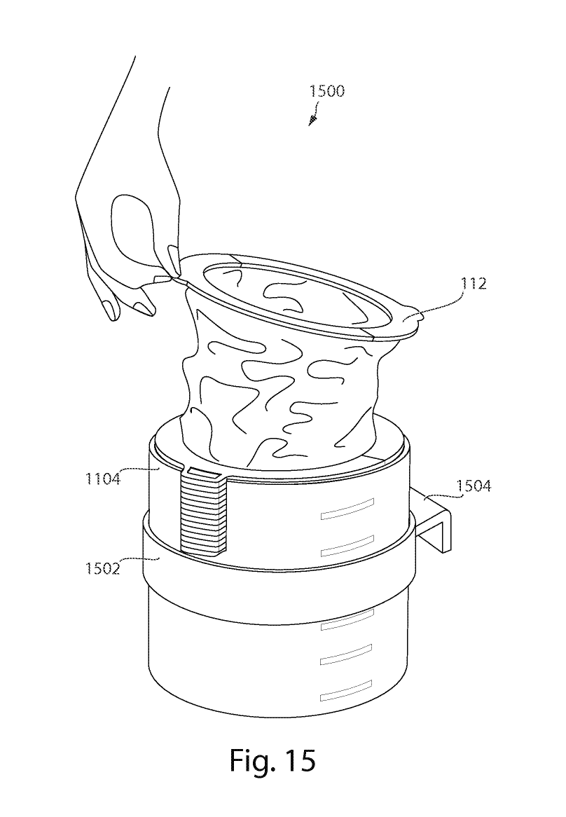

In an embodiment, the lid 1108 may have a lid opening, as seen in FIG. 14, such that the top most bag may be filled without removal of the lid 1108. The lid opening may be created during manufacturing, removal of a portion of the lid 1108 such as by tearing along a perforation, peeling away a cover material, and the like. The lid opening may expose only the bag opening while the remainder of the lid 1108 may protect the frame from contamination. After filling the top most disposal bag, the lid 1108 may be opened, removed, or otherwise moved out of the way in order to expose the filled bag such that the filled bag may be removed and sealed for ultimate disposal. Once the bag containing the disposed items may be removed, the next bag may be available for use. In an embodiment, the lower portion of the container may not be sized to allow the disposal bags to hang such that the bags may be filled while supported in the container. The bags may need to be removed one at a time prior to use. Alternatively, all of the bags may be removed and placed into a larger container for storage and use as described herein.

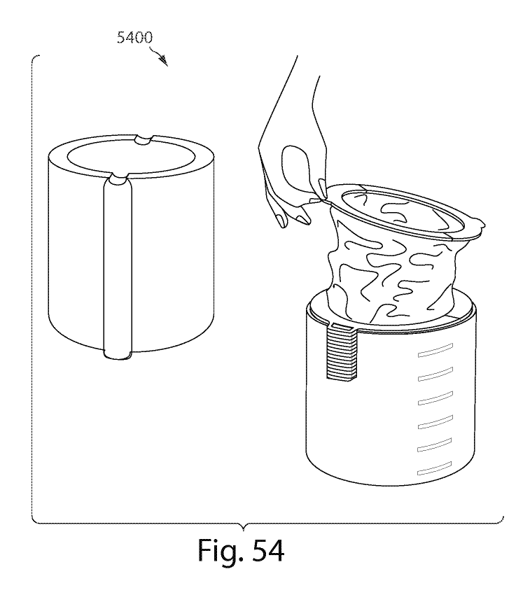

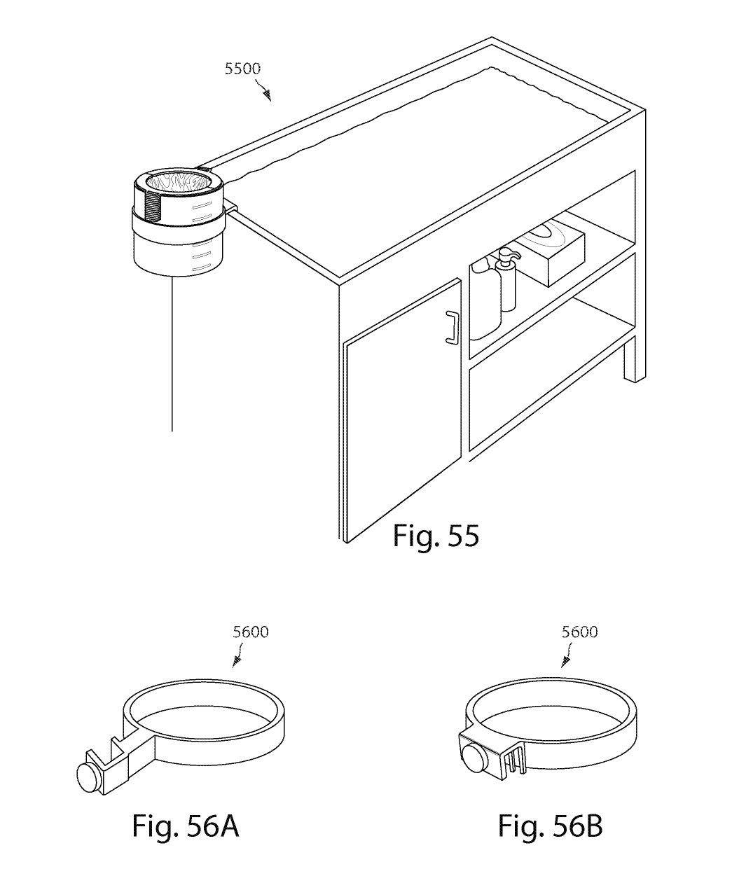

In an embodiment, the disposal system may comprise a mounting system. For example, the mounting system may be a shaped wire 1502 that may attach to an object, such as the rear of a car seat, a wall, a crib, and the like. The wire 1502 may be shaped so that the diameter of the shape may be smaller than the diameter of a frame such that a frame may rest upon the wire. The bags may be protected from contamination by a lid. For example, a mounting device 1502 may encircle a container to snugly hold it in position while an adjustable clamp 1504 firmly attaches the device to a piece of furniture such as a baby changing table, as in FIG. 55, a kitchen counter, a work surface, a piece of furniture, and the like. Other securement options may include hook-and-loop, adhesives, snaps, suction cups, and the like. A fabric strap-like material, optionally with further securing features or textures such as zip-tie or hook-and-loop material may assist in securing the system around an irregular or over-sized object. In any event, the mounting system may be modular and may provide for both vertical and horizontal clamp attachment in various forms, as in FIG. 56. The encircling portion of a mounting system may be common to several mounting geometries, but may also be accomplished by a feature that may be part of the container itself and therein a direct connection to the clamping portion of the mounting system. In the case of mounting to a horizontal element, there may be provided a U-shaped clamp body that may accommodate a wide range of dimensions for a horizontal edge feature and a clamping screw driven by a knob that may tighten firmly onto that element. In the case of mounting to a vertical element, there may be provided a U-shaped clamp body that may accommodate a wide range of dimensions for a vertical feature and a clamping screw driven by a knob that may tighten firmly onto that element.

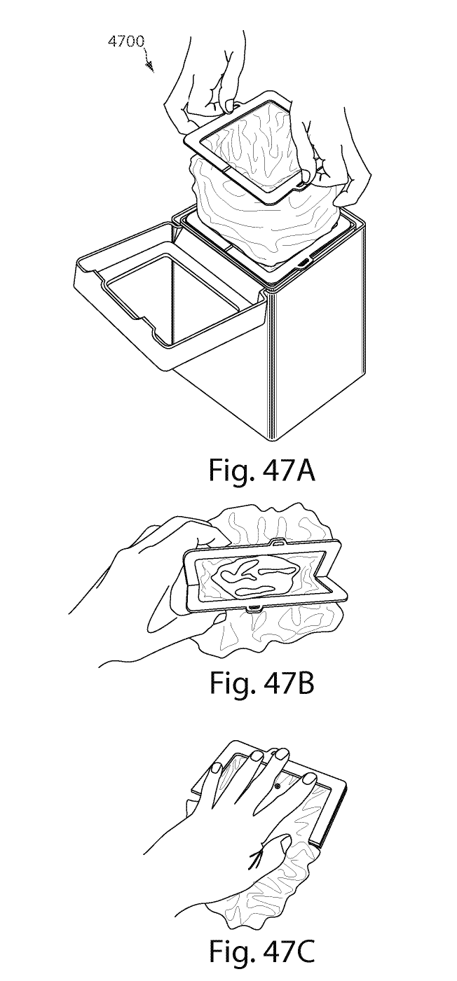

Referring now to FIG. 11, a waste disposal system 1100 may comprise a disposal bag 1112, a container 1104, waste clean-up articles 1118, and other materials that may facilitate disposal and/or clean-up of waste. A dispenser for waste clean-up articles 1114 may be integrated into the disposal bag container 1104. For example, a tissue dispenser 1114 may be integrated into the lid 1108 of a container 1104. The dispenser 1114 may be located on an exterior surface of the lid 1108 or an interior surface of the lid 1108. In another example, in FIG. 48, waste clean-up articles are dispensed in a container disposed in the lid of the disposal bag container. The waste clean-up article container extends into the opening of the disposal bags when the lid of the disposal bag container is replaceds. The tissues 1118 may be wet, such as for a baby wipe, or dry. The tissues 1118 may be used to wipe, wash, disinfect, cleanse, and the like. When the tissue dispenser 1114 is mounted on an interior surface of the lid, the dispenser 1114 may extend into a cavity created by the stacked disposal bag openings 1102. The tissue dispenser 1114 may have a removable lid with an orifice that may permit the tissues 1118 to be sequentially pulled out one at a time. The last pulled tissue 1118 may engage the next in line to present an edge through the opening. The waste disposal system 1100 may be intended to match the number of tissues 1118 to the number of disposal bags 1102 or may match the number of tissues 1118 and bags 1102 in some other proportion. The tissue dispenser 1114 may be refillable. Other materials that may be conveniently included in the waste disposal system 1100 may be one or more of hand sanitizers, skin moisturizers, creams, lotions, rash medication, and the like. These materials may be included in packets similar to disposable condiment packets.

In an embodiment, as shown in FIG. 72C, the frame 112 with a conical section may enable nesting of disposal bags to form a stack of nested bags 7204. For example, nesting may be where the conical shaped frame of a first disposal bag may provide support on its upper surface for the conical shaped frame of a second disposal bag when a second bag is positioned on top of the first bag such that the lower surface of the conical shaped frame of the second frame loosely fits into the upper surface of the conical shaped frame of the first frame and the second bag extends into the first bag. A plurality of bags may be successively nested to form the stack of nested bags 7204 and the frames 112 may be successively stacked, one upon the other. The stack of nested bags 7204 may provide a tighter nesting as compared to the stack of nested bags 1102, as described earlier. Further, the stack of nested bags 7204 may be contained in a container 7202 for storing and dispensing the bags. The container 7202 may facilitate a user using and/or pulling out one bag at a time. In one embodiment, the container 7202 may be similar to the container 1102 in design and operation.

In another embodiment, the container 7202 may be such that it has openings at top as well as bottom, similar to a pipe and can be of any suitable cross-section depending on the shape and size of the stack of nested bags 7204. Further, the container 7202 may have a flange inside the container 7202 near the bottom opening of the container 7202 to support the stack of nested bags 7204. The flange may be such that it should not tear-off the bag, when a user pulls out a bag from the bottom opening of the container 7202.

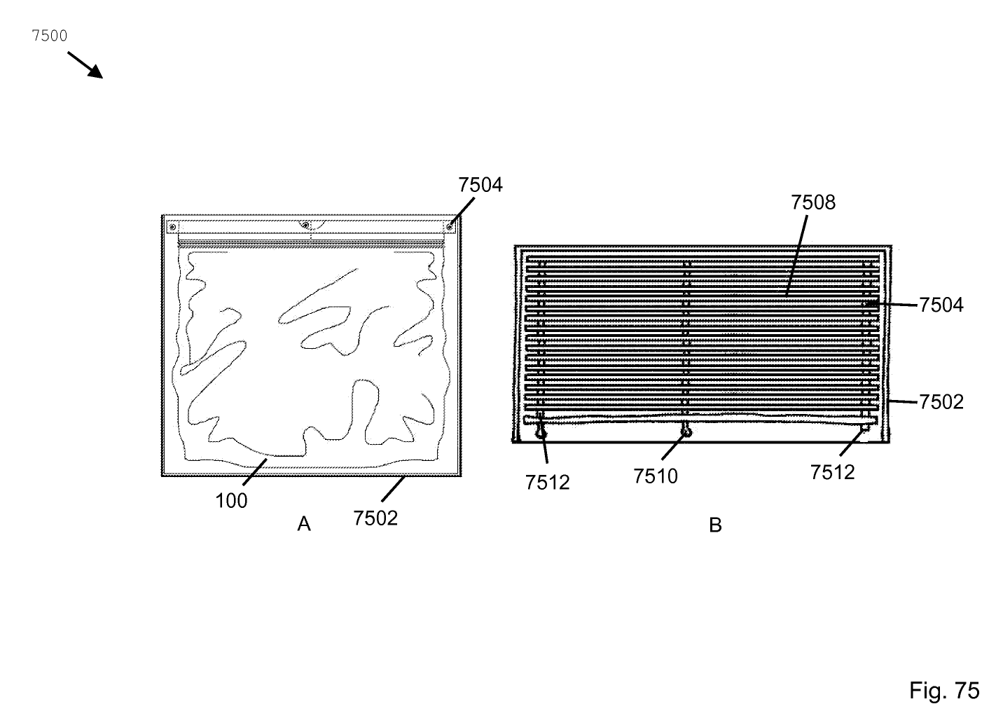

In embodiments, as depicted in FIGS. 75A and 75B, the disposal bags may be stacked in a dispensing container 7502 to form a stack of disposal bags 7508. The dispensing container 7502 may include wires/prongs 7504 to ensure easy and proper stacking of the disposal bags in the dispensing container 7502. The disposal bags may be stacked on the wires/prongs 7504 with the help of holes 7318 on the end tabs 7314 of the disposal bags. For example, a first disposal bag may be placed in the dispensing container 7502 by running the wires/prongs 7504 through the holes 7318 of the first disposal bag. After the first disposal bag has been inserted in the dispensing container 7502 and is resting on the wires/prongs 7504, more disposal bags may be inserted one-by-one in a similar manner to obtain the stack 7508. Further, the stack 7508 may be secured by an enlarged end 7510 of the wires/probes 7504. The enlarged end 7510 may ensure that the disposal bags in the stack 7508 come out of the dispensing container 7502 only when a force is applied to move the holes 7318 past the enlarged ends 7510. The combination of the enlarged ends 7510 and holes 7318 may comprise and interference locking system for stacking the disposal bags. It should be understood that other interference locking systems may be used in generating and maintaining a stack of disposal bags.

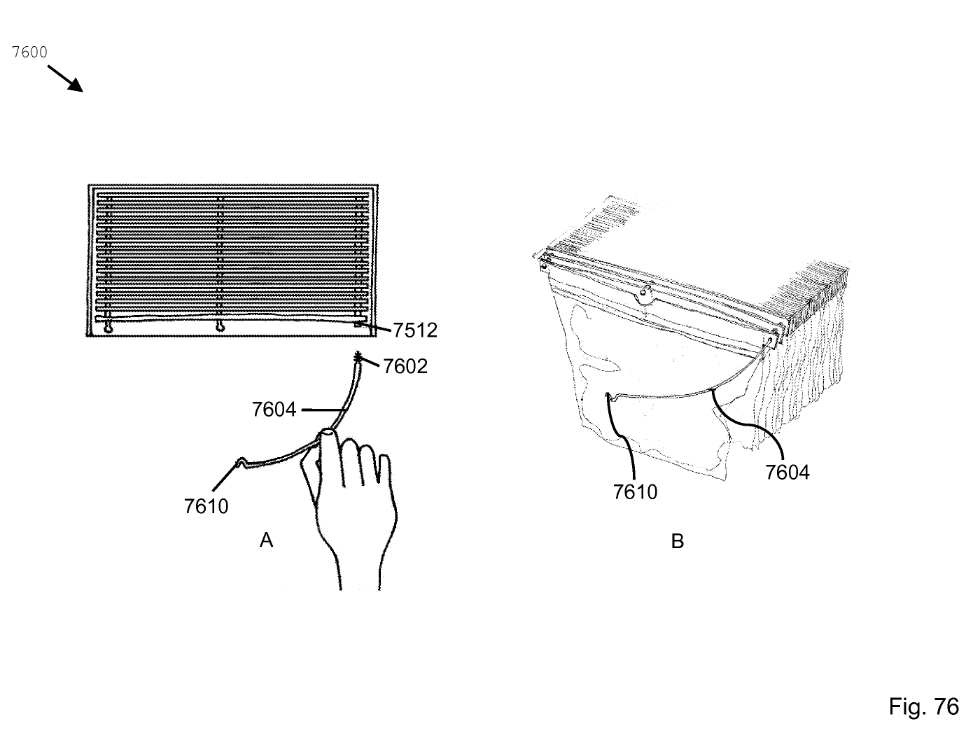

After the disposal bags have been assembled to form the stack 7508, the disposal bags may be pulled out of the dispensing container 7502 by the user. The disposal bags may be pulled out using a cutout 7320. The cutout 7320 may provide a finger grip and may also provide clearance for an optional middle hole. In an embodiment, an extension element 7604 may be provided to assist the user in pulling out the disposal bags from the dispensing container 7502. The extension element 7604 may be a curved-shaped wire such that it makes the disposal bag easy to open with a single-handed operation. The extension element 7604 may be connected to one of the wires/prongs 7504, as shown in FIG. 76. The connection may be made by attaching an engagement feature 7512 of the wire or the prong to an attachment feature 7602. The extension element 7604 may also have an end wire feature 7610 that may secure the tab of the disposal bag at the full open orientation.

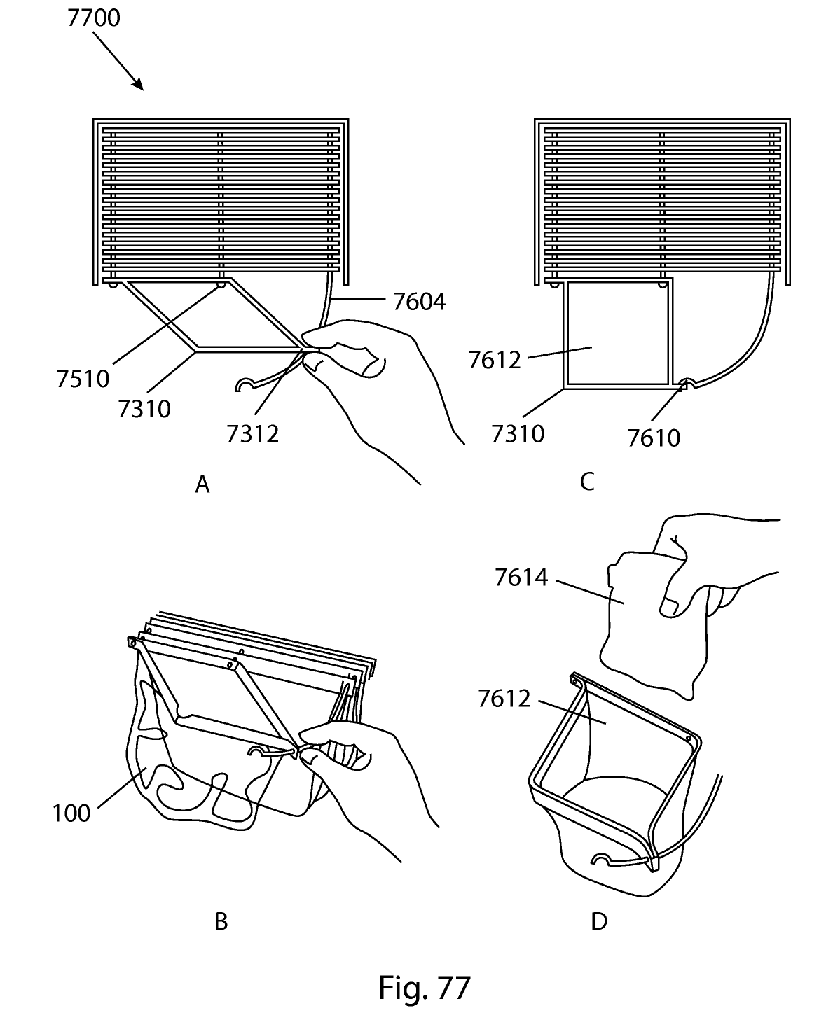

The user may pull a disposal bag 100 from the stack 7508 by pulling the tab 7314 with his/her fingers. As the user pulls the tab 7314 along the curved portion of the extension element 7604, the disposal bag 100 opens up, as shown in FIG. 77A and 77B. The frame 112 of the disposal bag 100 may take the shape of a parallelogram during this step. Further, the enlarged end 7510 of the wires/probes keep the rest of the disposal bag 100 located against the dispensing container 7502 during this step.

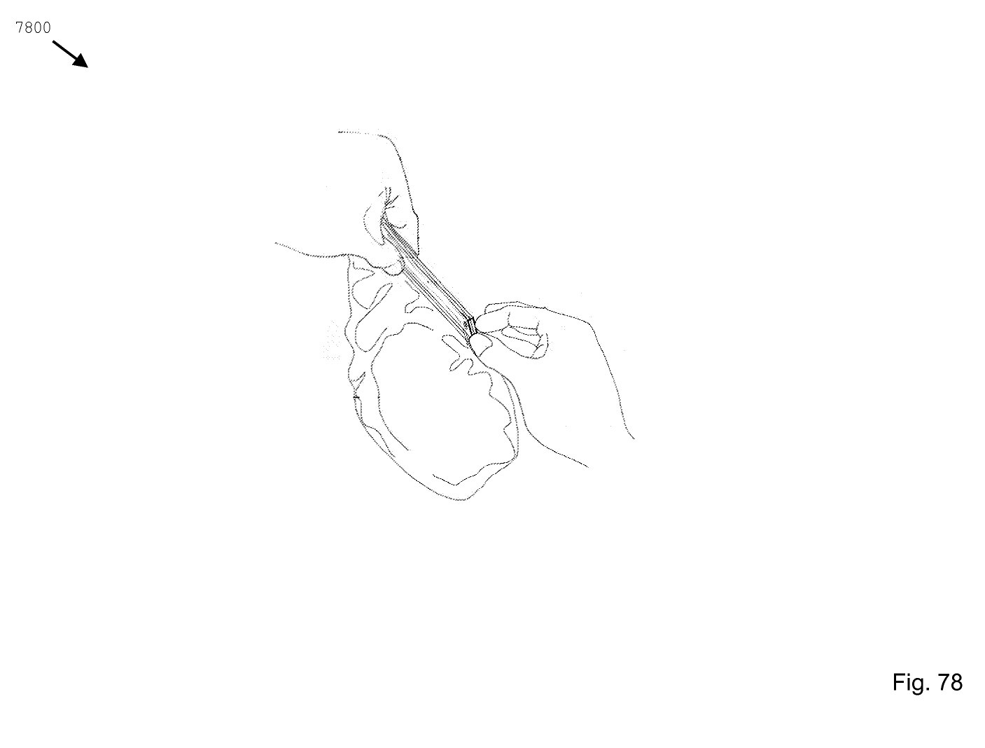

Now, as the disposal bag 100 opens up, the hinge points 7310 and 7312 achieve a configuration where they are bent, such as at ninety degrees. At this configuration, as shown in FIG. 77C and 77D, the disposal bag 100 may be fully open and may provide the largest possible opening 7612 of the disposal bag 100. Once the disposal bag 100 fully opens up, the tab 7314 may be secured by the end wire feature 7610 of the extension element 7604. Following this, waste material 7614 may be placed in the disposal bag 100. Once the disposal bag 100 is full of the waste material 7614, it may be easily removed from the extension element 7604 and the enlarged end 7510. Further, the disposal bag 100 may be sealed, as shown in FIG. 78.

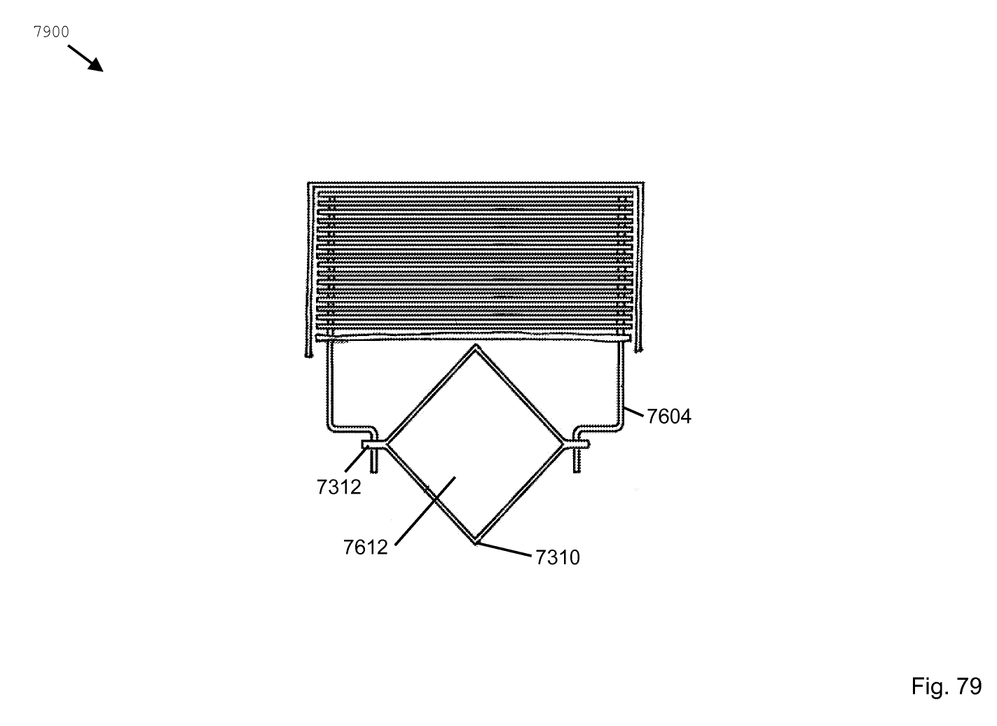

In embodiments, the extension element 7604 may have other geometries, as depicted in FIG. 79. In these embodiments, two-handed operation may be required in moving the tabs 7314 on either side of the frame 112 to an inner position on the extension element 7604 that bends the hinge points 7310 and 7312 to ninety degrees to produce the largest possible opening 7612 for the disposal bag 100.

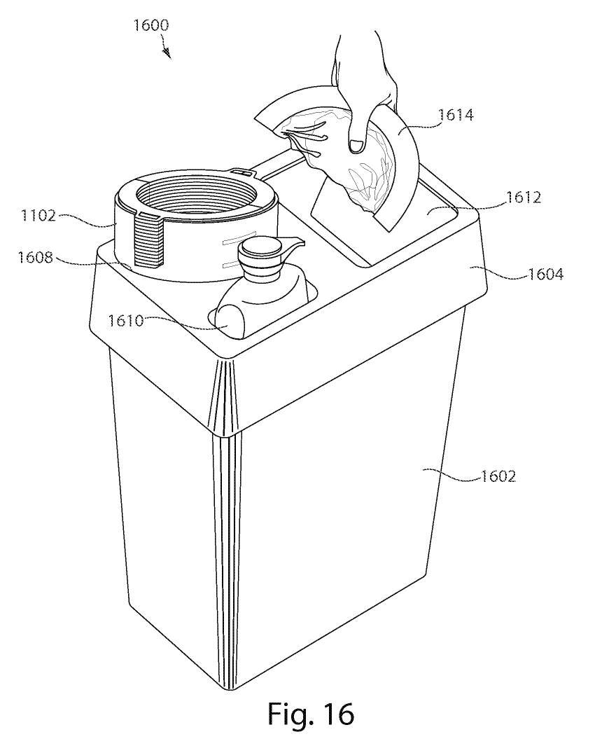

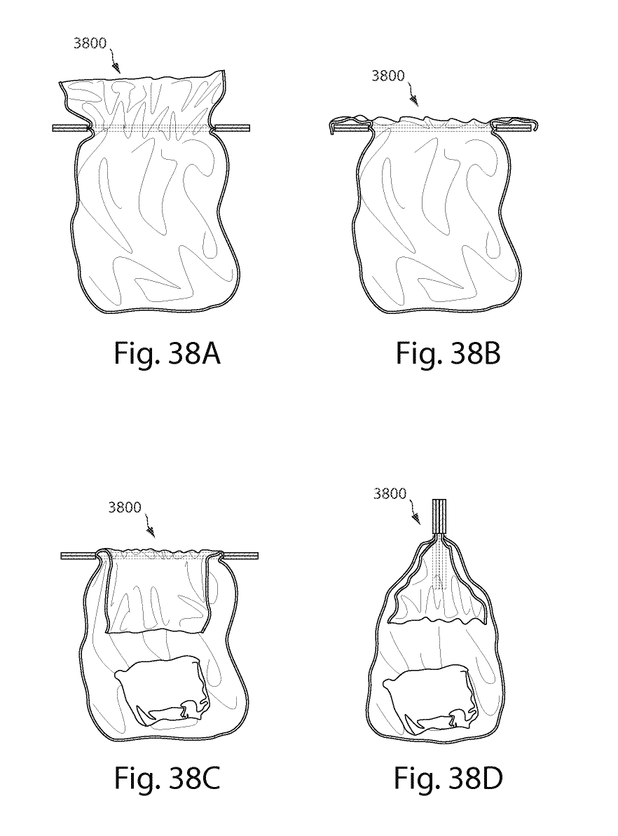

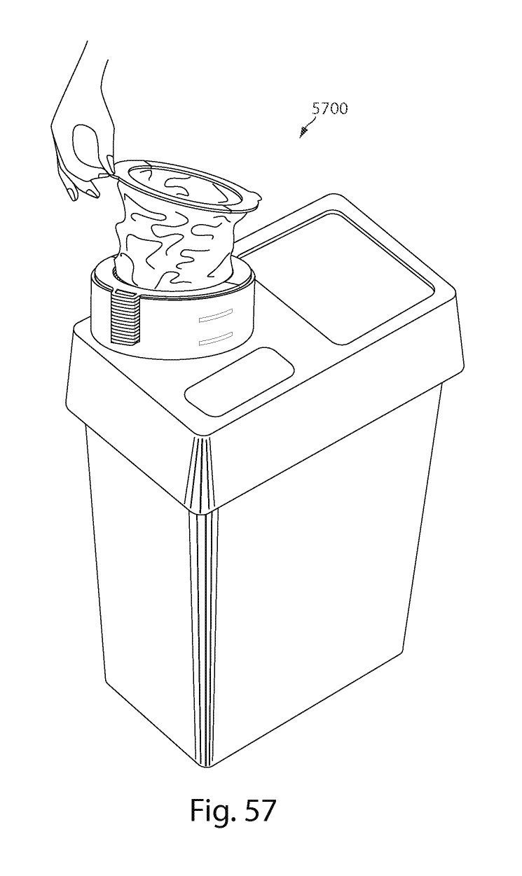

In an embodiment, the waste disposal system 1600 may be integrated in or attached to a secondary waste container 1602, as in FIG. 16 and FIG. 57. For example, a secondary waste container lid 1603 may have an orifice 1608 of a diameter that is slightly smaller than the diameter of a frame 112 such that a stack of nested bags 1102 may be inserted in the orifice 1608 and the stacked frames may rest upon the secondary waste container lid 1604. The nested bags 1102 may extend into the secondary waste container 1602. In another example, a secondary waste container lid 1604 may have an orifice 1608 that may accommodate a container 1104 of nested bags 1102 as described herein. The secondary waste container lid 1604 may also comprise an orifice 1610 to insert a tissue dispenser, a bottle of lotion, a tube of cream, and the like. The secondary waste container lid 1604 may have a built-in tissue dispenser that may be refillable. The secondary waste container lid 1604 may have an opening 1612 to insert trash, including sealed, waste-containing disposal bags 1614. The opening 1612 of the secondary waste container 1602 may have a cover. The cover may be hinged. The cover may be opened by any mechanical means such as a spring-loaded release mechanism, a push, a pull, and the like. The cover may be opened by a motion detection system. Auxiliary items, such as cleaning wipes, lotions, creams, medications, and the like, may also be supported or included in the secondary waste container. For example, the lid 1604 or top portion of the secondary waste container may be adapted for supporting, holding, containing, or including the auxiliary items and primary disposal bags. A larger, secondary disposal bag may be contained within the secondary waste container 1602 for receiving the smaller, primary disposal bags, as seen in FIG. 40. Alternatively, the disposal bags may be of a larger size, such as to fit a household trash container. The trash container may be adapted to receive the larger size disposal bags. The lid 1604 or top portion of a secondary waste container 1602 with the features described herein may be provided for mounting to an existing secondary waste container, such as a trash can. Alternatively, a complete system for disposal 1600 including a secondary waste container with the features described herein may be provided. Alternatively, the disposal bags may be of a larger size, such as to fit a household trash container. The trash container may be adapted to receive the larger size disposal bags.

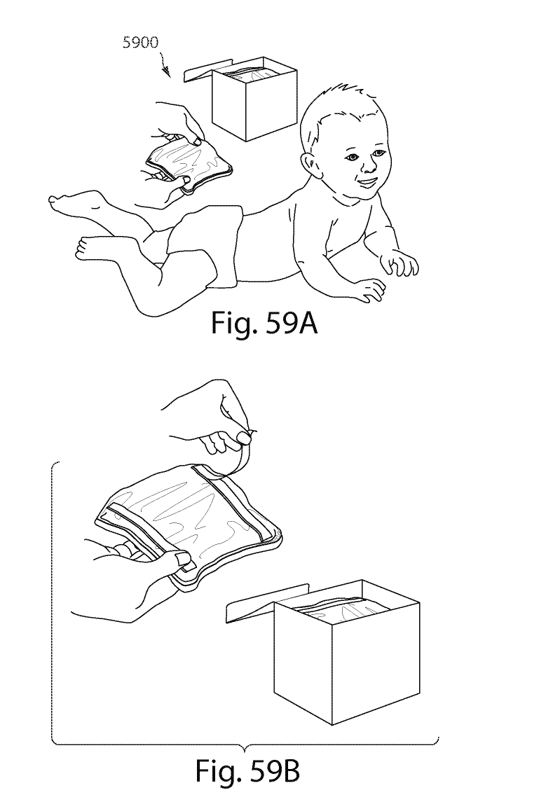

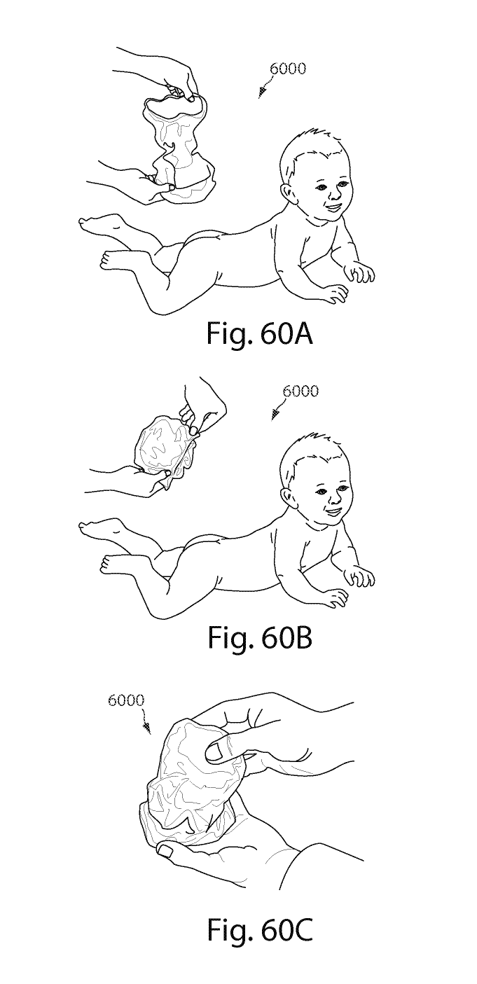

In an embodiment, as shown in FIG. 59, the disposal bag may be attached to the object to facilitate the object's disposal. When the object is ready to be disposed, the disposal bag attachment may be opened to reveal the disposal bag. In the example of the diaper, the open disposal bag may be opened to resemble a parachute-like structure, as in FIG. 60A, to encircle the diaper and contain the diaper contents within the disposal bag, as in FIG. 60B. The disposal bag may be sealed as described herein, as in FIG. 60C.

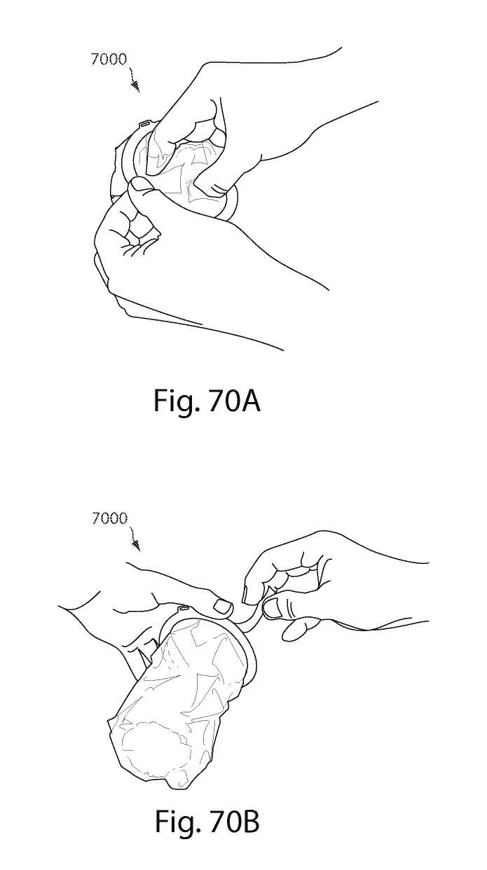

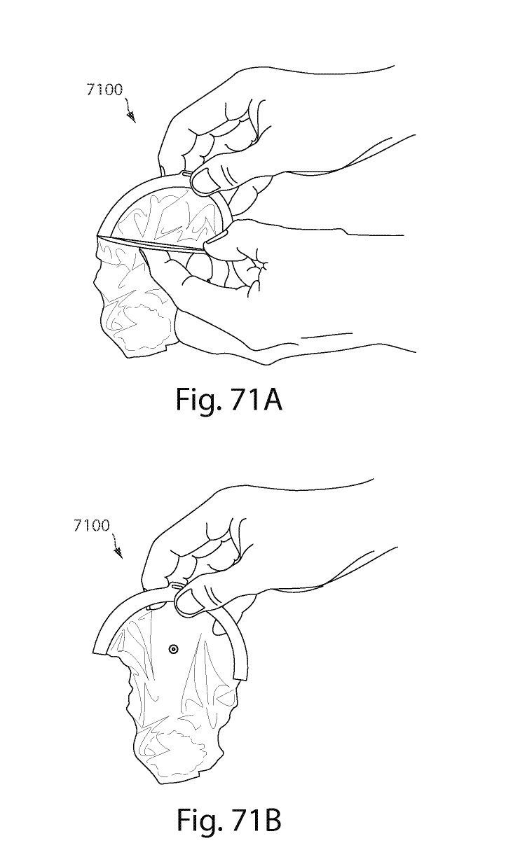

In an embodiment, the disposal bag may be wearable. For example, the disposal bag may be configured to be worn on a hand 1802. For example, the bag may be configured to be sealed in the inside-out configuration. Grasping and handling waste may be facilitated by fingered extensions of the bag, such as the fingers of a glove. The object may be grasped with the mitten or glove-like bag more easily because of the freedom of movement of the hand and fingers within the bag. In addition, having a glove-like bag may permit more secure grasp of a specimen within the bag. The user may then grasp waste 1808 with the inside-out bag 1802 and then invert the bag to remove it from their hand by pulling the open end of off the hand such that the waste remains in the interior of the bag, as shown in FIGS. 69 and 70, and a clean side of the bag is now exposed. A user may grasp waste from the inside with the finger tips then pull the open end of the bag off the arm and hand. The user may keep a hold of the closed end of the bag until it is safely pulled inside-out and the specimen or collected waste is securely inside (which was previously outside) of the bag. In some embodiments, the user or another person (e.g. a surgical assistant) may grasp the open end of the glove bag, then with the user's fingers pinching or restraining the closed end of the bag, pull the open end of the bag down over the user's hand so that the bag is inside out. The disposal bag 1802 may then be sealed as described herein and depicted in FIG. 71.

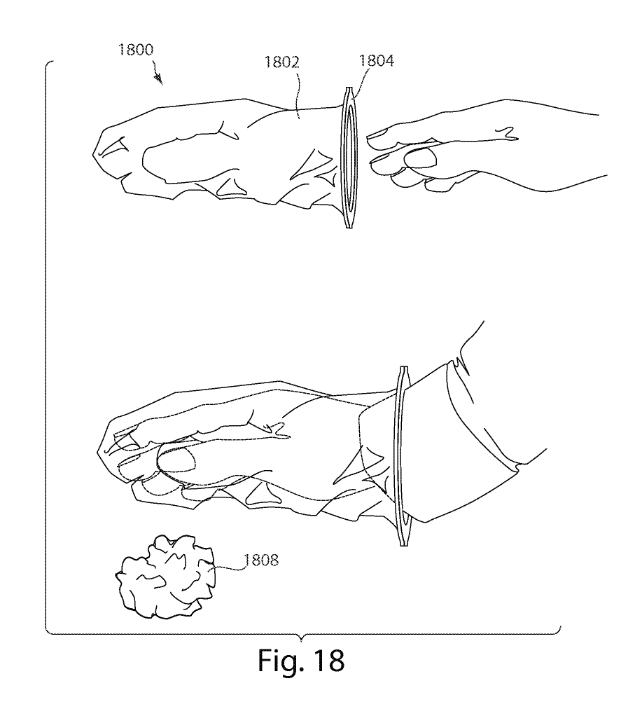

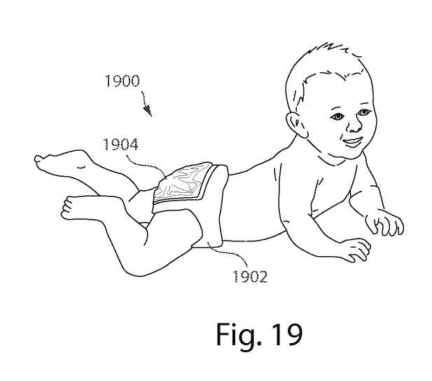

In embodiments, this embodiment of the disposal bag may be part of a waste disposal system such as described herein. For example, the disposal bag may be dispensed from a container or may be attached to an object, such as a diaper (as in FIG. 19 and FIG. 59) or sanitary napkin, for facilitating the object's disposal. As described herein, other components of such disposal systems may include a dispenser that dispenses a plurality of disposable glove bags such as the one shown in FIGS. 18, 69, and 70, sanitary wipes for cleaning and freshening, and a waste canister for containing the glove bag after use. As shown in FIG. 69A, the glove bag may be formed or cut in the shape of a hand, similar to the shape of a mitten or a glove to suit the wearing of the glove bag and to allow for natural movement and operation of the fingers and better grasping capability. The material from which the glove bag is made may vary depending on the specific use. Typical materials may be plastic, poly, pliable sheet material, or other material suitable for handling and storing the waste being disposed. The bag material may be leak-proof or leak-resistant to minimize or prevent spilling or leakage of liquids and/or gases from within the bag once it is sealed. Industrial or medical or radioactive applications might call for a heavier grade bag material than diaper changing or food preparation bags because of the dangers associated with tears and punctures of the bag in the former applications.

In an embodiment, the material might only be semi-permeable. For example, if dry solid materials only are being handled, then the bag body may be formed from paper or fabric or another material that can pass gas or liquid but not solid material. The glove bag may have a closed end, nearer to the finger tips, and an open end, similar to a conventional glove or mitten in this regard. However, the present wearable enclosure glove bag may also have a sealing mechanism or means near the open end thereof. In the examples shown in FIGS. 18, 69, and 70, the glove bag may include a collar flange at or near its open end that provides the means for sealing off the open end of the glove bag. The collar flange can be cardboard, heavy plastic, or other suitable material. The collar flange, similar to the frame in other embodiments, may be integrally formed along with the glove bag body or may be formed by joining a separate sealing flange to the portion of the glove bag body near the open end. Epoxy, bonding material, adhesive, heat, ultrasonic welding, chemical, mechanical, or other means may be used to join the glove bag body and the sealing flange to each other. The bond between the glove bag body and the flange may be leak-tight to the extent the bag is intended to resist leaks after use. The collar flange may be hinged so that the bag opening closes when the two portions of the flange along the hinge- or crease-line are brought into contact with one another. In some embodiments, a zipper style closure may be provided on the perimeter of the bag so that once used and in the inside-out configuration, the interlocking zipper mechanism may be on the interior of the bag perimeter and ready for sealing by mating the two opposing stripes of the zipper mechanism. Similarly if the sealing means is an adhesive strip or pair of strips or ring, the user may remove a protective film from the adhesive strip(s) after the bag is removed and inverted to allow for pressing the open end of the bag shut to seal its opening.

Optionally, the glove bag may be formed from tubular stock material that may be cut or molded or blown or fashioned into a series of similar glove bags during the manufacturing process.

In one or more embodiments, as in FIG. 69B, excess bag body material may allowed to extend out of the open end of the bag to protect the sleeves of the user, but can then be crumpled back into the volume of the bag after use. This may be done by crimping or pinching a portion of the bag body sleeve region between two flange material disks. In use, the user may wear the glove bag in an orientation that leaves a sealing face of the collar flange properly oriented for sealing after use. The user may then approach and grasp the object being picked up, handled, or processed.

In embodiments, the body of the glove bag may include a release facility that may allow for deliberate pressure on the sealed bag to expel excess air from within the sealed bag after use, as in FIG. 71B. The one-way vent may be in the form of a constriction or orifice or convoluted air passage that may require pressure to compress the used bag and eject the excess air from the vent while keeping the non-gaseous waste or samples inside the bag. Ejecting the excess gas from the bag may help store and dispose the bag with minimal volume usage.

One or both faces of the wearable enclosure glove bag may be treated with anti-bacterial agents, powder or other anti-perspirant or anti-frictional substances. Also, a fragrance, deodorizing substance or other mood- or atmosphere-enhancing chemical additive may be applied to reduce the effect of the objectionable contents of the bag on the user and his or her environment.

The glove bag may in some embodiments be opaque to prevent visual discomfort resulting from the appearance of the disposed glove bag. Also, this might protect the privacy of a user on which the glove bag was used in some applications. In other embodiments, the glove bag may be colored for aesthetic purposes or for functional purposes. For example, the bag body may be made of a recognizable colored film material that may indicate to the user and/or others its use or contents. For example, if an institution uses red-colored bags for disposing of biological waste, then the glove bag may be made of red plastic material to indicate the same.

The bags may be imprinted with words or symbols to indicate a variety of things. For example, the glove bags may include at least a portion thereof on which a user can write before or after use to alert others to the contents of the bag, its date of use, or other information. In another example, in criminal or forensic investigations, the user might mark the bag with indelible ink with a date of use and a text or serial number indicating the source of the sample taken within the bag. Swipes of suspected areas can thus be accomplished and indexed or taken for later analysis at a central lab or station.

The glove bags may be in some embodiments made of imprintable material. For example, if prepared prior to use, the glove bags may be prepared on a sheet that permits the entire sheet and attached bag to be run through a printer for printing of information such as a serial number, batch number, warnings, and the like. The bags may be imprinted with a corporate logo or a recognized symbol. For example, a bag used for disposal of biological hazardous materials may be red with white lettering “BIOHAZARD”, with or without the recognized symbol for bio-hazard. In another example, a bag used for disposal of radioactive waste materials may be yellow with black lettering “RADIOACTIVE WASTE”, with or without the recognized symbol for radiation. In another example, a bag made for picking up animal waste may be of any opaque material and imprinted with a animal-like figure or other entertaining marking to increase the appeal of the product and warn of its contents. Those skilled in the art may appreciate that the glove bag may be used for applications such as collecting natural specimens, such as flowers, fruit, plants, insects, animals, sea creatures, soil, and the like, that persons on natural research and exploration missions on this planet or beyond may make use of to seal and return their specimens in.

Referring to FIG. 19, the disposal bag 1904 may be attached to a diaper 1902. The disposal bag 1904 may be attached to a diaper 1902 on an exterior surface that may not touch skin. The disposal bag 1904 may be secured to the diaper 1902 with adhesive such as glue, heat-activated polymer, double stick tape, microencapsulated adhesive, and the like. The adhesive may be protected by a peel away protective layer that may be peeled away to expose the underlying adhesive and prepare the disposal bag 1904 for securing to a diaper 1902. The disposal bag 1904 may be secured to the diaper 1902 with a mechanical coupling such as snaps, zippers, zip-tight style connections, spot welds, perforated seams, stitching, hook-and-loop fastening patches, mechanical fasteners, safety pins, and the like. A cleaning wipe or cloth or tissue may be folded and inserted into the disposal bag 1904 to be used in wiping a child when changing the diaper 1902. The used wipe may be stuffed into the disposal bag 1904 along with the diaper 1902. Hand cleaning cloths, sanitizing napkins, diaper rash creams, moisturizers, ointments, scented fluids, and the like may be carried along with or included in the package. For example, a skin ointment may be provided in a single-use package and may be attached to the diaper 1902 or contained within the disposal bag 1904. All of the used waste and packaging may be placed inside the disposal bag 1904 and stored or discarded upon use. The disposal bag 1904 may remain attached to the diaper 1902 and inverted onto the used diaper 1902 for disposal. The disposal bag 1904 may be completely detached from the diaper 1902 and the diaper 1902 may be placed into the bag 1904 after it is detached. As described herein, a user may invert the disposal bag 1904 and place it upon a hand in order to reach through the bag 1904 and grasp the diaper 1902 and waste. The diaper 1902 and waste may then be pulled into the bag 1904. In some embodiments, the disposal bag 1904 may be configured to be or may be contained in a flat pack. The flat pack may be accessed via tab or other graspable feature that may enable opening of the flat pack to deploy the disposal bag 1904 to prepare it for receiving the used diaper 1902 and waste. The bag 1904 may remain attached to the diaper 1902. Deployment of the disposal bag 1904 may be similar to the unfurling of a parachute. The disposal bag 1904 stored in the flat pack may have a thin dimension and may be stacked in large numbers in a container for distribution, sales, on-site storage, and selection. The disposal bag 1904 may further comprise a frame 112. The frame 112 may be sealed as described herein. Alternatively, the disposal bag 1904 may be sealed by one or more of a formed plastic zipper mechanism, an attached tie material such as a strip of plastic, string, or fabric, microencapsulated adhesive, an adhesive tab, a knot, and the like.

In an embodiment, the disposed waste may be baby diapers, incontinence products, feminine products, pet waste, kitchen waste, household waste, garage or automotive waste, paint- or oil-saturated rags, medical waste, biohazardous and hazardous material, industrial shop waste, camping waste, waste generated in a car or during travel, radioactive materials, other materials or waste with an objectionable odor, and the like.

It may be appreciated by one of skill in the art that the present invention may be applied in the storing, keeping, sealing, preserving, collecting and the like of non-offensive materials, and may in some cases be used to hold, seal, collect, and preserve fresh goods, food, samples, and the like.

Many other systems, methods, object, features, and advantages of the present invention will be appreciated. All such systems, methods, object, features, and advantages are within the scope of the present disclosure.

While the invention has been disclosed in connection with certain preferred embodiments, those of ordinary skill in the art may recognize other embodiments, and all such variations, modifications, and substitutions may be intended to fall within the scope of this disclosure. Thus, the invention may be to be understood in the broadest sense allowable by law.

What is claimed is:

1. A disposal kit comprising:
a bag attached to a wearable diaper, the bag being configured to receive waste;
a frame attached to the bag at an opening, the frame being configured to enable a user to close the bag in a single-handed operation when opposing sides of the frame are brought into contact with one another; and
a sealing facility disposed on the frame for providing a resealable closure of the bag when the opposing sides of the frame are brought into contact with one another.

2. The disposal kit of claim 1 wherein the frame is hinged.

3. The disposal kit of claim 1 wherein the sealing facility is at least one of an interference locking system, a tab, discrete interlocks, a snap fit, and a clasp.

4. The disposal kit of claim 1 wherein the sealing facility is an adhesive.

5. The disposal kit of claim 1 further comprising, a gasket mounted to the frame.

6. The disposal kit of claim 1 further comprising, a release facility disposed on the disposal bag.
